(19) United States
(12) Patent Application Publication
(10) Pub. No.: US 2026/0060328 A1
(43) Pub. Date: Mar. 5, 2026

(54) AEROSOL-GENERATING DEVICE

(71) Applicant: Philip Morris Products S.A., Neuchatel (CH)

(72) Inventors: Johnny GRZAN, Hong Kong (HK); Eric MARIACHER, Jougne (FR); Guoqiang CAI, Shenzhen (CN); Hongjie XU, Hong Kong (HK); Zhuowen LIN, Shenzhen (CN)

(73) Assignee: Philip Morris Products S.A., Neuchatel (CH)

(21) Appl. No.: 19/105,145

(22) PCT Filed: Sep. 2, 2022
(86) PCT No.: PCT/CN2022/116794

§ 371 Date: Feb. 20, 2025

(51) Int. Cl. A24F 40/57; A24F 40/53; A24F 40/60; A24F 40/90; A61B 5/117; H01M 10/44; H01M 10/48

(52) U.S. Cl. CPC A24F 40/57; A24F 40/53; A24F 40/60; A24F 40/90; H01M 10/44; H01M 10/488; A61B 5/117; H01M 2220/30

(57) ABSTRACT

An aerosol-generating device is provided, including: control circuitry and an energy storage configured to supply electrical energy to the control circuitry for generating aerosol from an aerosol-generating article; the control circuitry being configured to: determine a storage status of the energy storage indicative of at least one of an amount of electrical energy currently stored and currently storable in the energy storage, evaluate the determined storage status with respect to at least one threshold value correlating with a threshold energy required for performing a main heating function of the device for heating the article at or above a predetermined heating temperature to generate aerosol in at least one usage session, and for performing at least one auxiliary device function of the device different than the main heating function, and enable or disable, based on the evaluation, at least one of the main heating function and the auxiliary device function.

The present disclosure generally relates to the field of aerosol-generating devices and systems for generating aerosol. In particular, the present disclosure relates to electronic aerosol-generating devices and systems configured to generate aerosol based on heating at least a part or portion of an aerosol-generating article or substrate. The present disclosure further relates to use of one or more aerosol-generating devices or systems, to methods of operating aerosol-generating devices or systems, to corresponding computer programs and to corresponding computer-readable media storing one or more of such computer programs.

Aerosol-generating devices are typically designed as handheld devices that can be used by a user for consuming or experiencing, for instance in one or more usage sessions, aerosol generated by heating an aerosol-generating substrate or at least a part of an aerosol-generating article comprising such substrate. It will be appreciated that aerosol-generating devices can generate aerosol by other means, such as for example by vibrating, by spraying or other means.

Exemplary aerosol-generating substrates can comprise solid substrate material, such as tobacco material or tobacco cast leaves (“TCL”) material. The substrate material can, for example, be assembled, often with other elements or components, to form a substantially stick-shaped aerosol-generating article. Such a stick or aerosol-generating article can be configured in shape and size to be inserted at least partially into the aerosol-generating device, which, for example, can comprise a heating element for heating the aerosol-generating article and/or the aerosol-generating substrate. Alternatively or additionally, aerosol-generating substrates can comprise one or more liquids and/or solids, which can for example be supplied to the aerosol-generating device in the form of a cartridge or container. Exemplary aerosol-generating articles can comprise a cartridge or container that contains or is fillable with the liquid and/or solid substrate, which can be vaporized during aerosol consumption by the user based on heating the substrate. Usually, such cartridge can be coupled to, attached to and/or at least partially inserted into the aerosol-generating device. Alternatively, the cartridge may be fixedly mounted to the aerosol-generating device and refilled by inserting liquid and/or solid substrate material into the cartridge.

For generating aerosol during use or consumption, a user typically actuates a user interface of the aerosol-generating device, thereby triggering supply of one or more aerosol-generating means or aerosol generators, such as one or more heating elements or heat sources, with electrical energy, for example to heat at least a portion of the aerosol-generating substrate or article. At least a part of the aerosol-generating means or aerosol generator, for example at least a part of the heating element, can be arranged in the aerosol-generating device. Alternatively or additionally, at least a part of the aerosol-generating means or aerosol generator, for example at least a part of the heating element, can be arranged in the aerosol-generating article.

Exemplary heating elements can be based on one or more of resistive heating, inductive heating and microwave heating using electrical energy supplied via, drawn from or stored in an energy storage of the aerosol-generating device. Exemplary energy storages can include one or more batteries, one or more capacitors, one or more supercapacitors, one or more accumulators or other types of energy storage.

Alternatively or additionally, the aerosol-generating device may be configured to supply electrical energy to one or more other aerosol-generating means, aerosol engines or aerosol generators to generate aerosol. For example, the aerosol-generating device and/or aerosol-generating article may comprise one or more vibrating elements, one or more vibrating meshes, one or more spraying devices, or other means for generating aerosol.

Generally, an amount of electrical energy provided by the energy storage and usable by or available to a user of the aerosol-generating device to operate the aerosol-generating device, for example to perform one or more device functions of the aerosol-generating device, may be limited, thereby potentially limiting functionality or usability of the aerosol-generating device to generate aerosol.

Further, limitations in capacity of the energy storage and/or frequent use of the device to generate aerosol in one or more usage sessions may result in frequent charging and discharging of the energy storage. Potentially, such frequent charging and discharging of the energy storage may adversely affect a quality of the energy storage and its capability to store electrical energy may potentially decrease over time. After a certain lifetime or service-life of the energy storage, the aerosol-generating device or at least the energy storage may be replaced.

It may, therefore, be desirable to provide for an improved aerosol-generating device or system, for example an aerosol-generating device or system overcoming or at least mitigating one or more of the aforementioned drawbacks.

This is achieved by the subject-matter of the independent claims. Optional features are provided by the dependent claims and by the description.

Aspects of the present disclosure relate to one or more aerosol-generating devices and one or more aerosol-generating systems configured to generate aerosol from at least a part of an aerosol-generating article or substrate, for example based on supplying one or more aerosol generators with electrical energy. The present disclosure further relates to use of such one or more aerosol-generating devices and/or systems, to methods of operating such one or more aerosol-generating devices and/or systems, to corresponding computer programs and to corresponding non-transitory computer-readable media storing one or more of such computer programs. Any disclosure presented hereinabove and hereinbelow with reference to one or an aspect of the present disclosure equally applies to any other aspect of the present disclosure.

According to an aspect of the present disclosure, there is provided an aerosol-generating device for generating aerosol. The aerosol-generating device comprises control circuitry and an energy storage configured to supply electrical energy to the control circuitry for generating aerosol from an aerosol-generating article. The control circuitry may be operatively coupled to the energy storage. The control circuitry is configured to determine a storage status of the energy storage indicative of at least one of an amount of electrical energy currently stored and an amount of electrical energy currently storable in the energy storage. The control circuitry is further configured to evaluate, for example process and/or analyse, the determined storage status with respect to at least one threshold value associated with at least one device function of the aerosol-generating device. The control circuitry is further configured to enable or disable, based on the evaluation, at least one device function of the aerosol-generating device. Therein, the at least one device function associated with the at least one threshold value may be enabled or disabled. Alternatively or additionally, at least one further device function, which may differ from the at least one device function associated with the at least one threshold value, may be enabled or disabled.

According to a further aspect of the present disclosure, there is provided an aerosol-generating device for generating aerosol. The aerosol-generating device comprises control circuitry and an energy storage configured to supply electrical energy to the control circuitry for generating aerosol from an aerosol-generating article. The control circuitry may be operatively coupled to the energy storage. The control circuitry is configured to determine a storage status of the energy storage indicative of at least one of an amount of electrical energy currently stored and an amount of electrical energy currently storable in the energy storage. The control circuitry is further configured to evaluate the determined storage status with respect to at least one threshold value, wherein the at least one threshold value correlates with a threshold energy required for performing a main heating function of the aerosol-generating device for heating the aerosol-generating article at or above a predetermined heating temperature to generate aerosol in at least one usage session, and for performing at least one auxiliary device function of the aerosol-generating device different than the main heating function. The control circuitry is further configured to enable or disable, based on the evaluation, at least one of the main heating function and the at least one auxiliary device function.

According to a further aspect of the present disclosure, there is provided an aerosol-generating device for generating aerosol. The aerosol-generating device comprises control circuitry and an energy storage configured to supply electrical energy to the control circuitry for generating aerosol from an aerosol generating article. The control circuitry may be operatively coupled to the energy storage. The control circuitry is configured to determine a storage status including a health status of the energy storage indicative of an amount of electrical energy currently storable in the energy storage, and to evaluate the determined storage status with respect to at least one threshold value associated with at least one device function of the aerosol-generating device. The control circuitry is further configured to enable or disable, based on the evaluation, at least one device function of the aerosol-generating device. Therein, the at least one device function associated with the at least one threshold value may be enabled or disabled. Alternatively or additionally, at least one further device function, which may differ from the at least one device function associated with the at least one threshold value, may be enabled or disabled.

According to a further aspect of the present disclosure, there is provided an aerosol-generating device for generating aerosol. The aerosol-generating device comprises control circuitry and an energy storage configured to supply electrical energy to the control circuitry for generating aerosol from an aerosol-generating article. The control circuitry may be operatively coupled to the energy storage. The control circuitry is configured to determine a storage status of the energy storage indicative of at least one of an amount of electrical energy currently stored and an amount of electrical energy currently storable in the energy storage. The control circuitry is further configured to evaluate the determined storage status with respect to at least one threshold value associated with at least one device function of the aerosol-generating device, wherein the at least threshold value is adjustable. The control circuitry is further configured to enable or disable, based on the evaluation, at least one device function. Therein, the at least one device function associated with the at least one threshold value may be enabled or disabled. Alternatively or additionally, at least one further device function, which may differ from the at least one device function associated with the at least one threshold value, may be enabled or disabled.

It is noted that any feature, function, or element of an aerosol-generating device according to one aspect of the present disclosure can be combined with any feature, function or element of aerosol-generating device according to another aspect of the present disclosure. This is the aerosol-generating devices according to various aspects of the present disclosure can be combined with one another. Also, any disclosure presented herein with reference to an aerosol-generating device according to an or one aspect of the present disclosure, equally applies to any one or more aerosol-generating device according to any one or more further aspects of the present disclosure.

In an example, the energy storage and/or control circuitry may be configured to supply electrical energy to at least one aerosol-generating means or at least one aerosol generator for generating aerosol from at least a portion of an aerosol-generating article couplable to the aerosol-generating device. Exemplary aerosol generators or means may include one or more heating elements, one or more heat sources, one or more vibrating elements, one or more vibrating meshes, and one or more spraying devices.

For example, the energy storage and/or control circuitry may be configured to supply electrical energy to at least one heating element to generate aerosol from an aerosol-generating article couplable to the aerosol-generating device, for example based on heating at least a portion of the aerosol-generating article.

As used herein enabling a device function may refer to or include allowing the device function, for example allowing execution of the device function, for example by a user of the aerosol-generating device. Alternatively or additionally, enabling a device function may include activating the device function, such that it can be performed, for example based on one or more user inputs or automatically. For instance, enabling or allowing a device function may include configuring the aerosol-generating device, such that the device function is executable or can be performed, for example by the user. Alternatively or additionally, enabling a device function may include making or rendering the device function available to the user of aerosol-generating device. In a non-limiting example, a flag or marker indicative of the device function being allowed may be set by the control circuitry based on the evaluation of the storage status with respect to the at least one threshold value in order to enable a device function.

As used herein disabling a device function may refer to or include preventing the device function, for example preventing execution of the device function, for example by a user. Alternatively or additionally, disabling a device function may include deactivating the device function, such that it cannot be performed, for example based on one or more user inputs or automatically. Alternatively or additionally, disabling a device function may include making or rendering the device function unavailable to the user of aerosol-generating device. For instance, disabling or preventing a device function may include configuring the aerosol-generating device, such that the device function and/or its execution is inhibited. In a non-limiting example, a flag or marker indicative of the device function being prevented may be set by the control circuitry based on the evaluation of the storage status with respect to the at least one threshold value in order to disable a device function.

Accordingly, enabling or disabling a device function may include altering, modifying or changing a configuration of the aerosol-generating device, such that the device function is available or unavailable at the aerosol-generating device, for example available or unavailable to a user of the aerosol-generating device. Alternatively or additionally, enabling or disabling a device function may include adapting a repertoire or set of device functions available at the aerosol-generating device. For instance, enabling a device function may include expanding the repertoire or set of device functions available at the aerosol-generating device by said device function. Alternatively or additionally, disabling a device function may include reducing the repertoire or set of device functions available at the aerosol-generating device by said device function. Alternatively or additionally, disabling a device function may include removing said device function from the repertoire or set of device functions available at the aerosol-generating device.

Evaluating the storage status of the energy storage with respect to the at least one threshold value and enabling or disabling at least one device function based thereon can allow for an energy management and efficient operation of the aerosol-generating device. In particular, operation of the device may be optimised based on adapting a repertoire or set of device functions available at the aerosol-generating device by evaluating the storage status. In other words, functionality of the device can be adapted in accordance with the evaluated storage status, which can allow to optimize or maximize overall functionality of the device with respect to the available energy. Also, functional versatility and flexibility of the device may be increased.

The aerosol-generating device of the present disclosure may particularly refer to an electronic aerosol-generating device configured to generate aerosol, in particular aerosol that can be inhaled by a user of the aerosol-generating device in or during one or more usage sessions based on heating at least a part of a heating element, at least a part of the aerosol-generating article and/or at least a part of an aerosol-generating substrate contained in the aerosol-generating article. The aerosol-generating device of the present disclosure may also be referred to as heat-not-burn device.

Exemplary aerosol-generating articles or substrates usable with the aerosol-generating device of the present disclosure can be stick-like formed and at least partly inserted into the aerosol-generating device. Alternative exemplary aerosol-generating articles can comprise a container or cartridge, which can be fixedly attached or detachably coupled to the aerosol-generating device. Typically, a liquid, solid or a mixture of solid and liquid aerosol-generating substrate is contained in or can be inserted into such aerosol-generating article and heated to generate aerosol. Any such as well as other forms and designs of aerosol-generating articles can be used with the aerosol-generating device and system of the present disclosure.

Generally, the control circuitry, also referred to as device control circuitry herein, may be configured to control one or more device functions of the aerosol-generating device. In particular, the device control circuitry may include one or more processors for data or signal processing. The device control circuitry may be configured to operatively control the aerosol-generating device, its energy storage and/or one or more further components of the aerosol-generating device. Operative control may involve controlling operation of the aerosol-generating device and/or one or more components thereof. Optionally, operative control of one or more device functions and/or of the energy storage may include generating and/or providing one or more control signals to one or more components of the aerosol-generating device, such as for example the energy storage.

As used herein, the energy storage of the aerosol-generating device may be configured to store or may store electrical energy that can be supplied to the at least one heating element to generate aerosol. Optionally, the energy storage may be re-chargeable, for example by connecting the aerosol-generating device to a power supply or a companion device, also referred to herein as receiving device, of an aerosol-generating system. For example, the energy storage may be re-charged by connecting the aerosol-generating device to the companion device or power supply via cable or inductive coupling.

It is noted that the aerosol-generating device can comprise a plurality of energy storages. Accordingly, any reference to a single energy storage hereinabove and hereinbelow includes a plurality of energy storages.

In an example, the energy storage may include one or more batteries, accumulators, capacitors, or other types of energy storage for storing electrical energy, such as for example an energy storage configured to store potential energy associated with a change in configuration of internal chemical elements or molecules of the energy storage. Also a combination of any of the aforementioned types of energy storages can be implemented in the aerosol-generating device of the present disclosure.

The at least one heating element may refer to or denote any one or more of an inductive heating element, a resistive heating element and a microwave heating element. In other words, the heating element can be configured to heat the aerosol-generating article based on one or more of inductive heating, microwave heating and resistive heating.

In an example, the heating element may be an inductive heating element, for example including an inductive coil, configured to inductively heat a susceptor or susceptor material arranged in the aerosol-generating article or substrate. Alternative or additionally, the heating element may comprise one or more heating blades or resistive heating elements which may at least partly be inserted into the aerosol-generating article or substrate, and supplied with electrical energy for generating aerosol. Alternatively or additionally, the heating element may include a microwave generator configured to heat the aerosol-generating article based on microwave heating. Other forms, such as loop gap resonators may be used in addition or as alternative.

At least a part of or the entire at least one heating element can be arranged in the aerosol-generating device. Alternatively or additionally, at least a part of or the entire heating element may be arranged in the aerosol-generating article. The aerosol-generating device may comprise a heating arrangement or heating circuit including at least one heating element. For example, a part of the heating element, circuit or heating arrangement may be arranged in the aerosol-generating device and a further part of the heating element, circuit or arrangement may be arranged in the aerosol-generating article. Further, it is noted that the aerosol-generating device and/or the aerosol-generating article can comprise a plurality of heating elements. Accordingly, any reference to a single heating element hereinabove and hereinbelow can include a plurality of heating elements.

In an example, the aerosol-generating device may comprise one or more user interfaces actuatable, controllable or operable by the user to activate or operate the aerosol-generating device to generate aerosol. Exemplary user interfaces may include a button, a switch, a touch display, an acoustic interface, a gesture control interface, a tactile interface, a haptic interface, or a combination thereof.

The control circuitry may be operable to receive and/or process one or more user inputs from the user interface and operate or power the heating element, respectively a heating circuit or arrangement including the heating element, to generate aerosol in accordance with the one or more user inputs. For instance, in response to an actuation of the user interface, electrical energy may be supplied to the at least one heating element to heat at least a part of the aerosol-generating article and generate aerosol.

As discussed above, the aerosol-generating device may comprise a repertoire or set of a plurality of device functions that can in principle be performed or executed by the aerosol-generating device, for example based on a user input and/or automatically. One or more of these device functions may be available to the user and/or may be performed or executed by the user. For example, the user may operate the aerosol-generating device and/or one or more user interfaces of the aerosol-generating device to perform one or more device functions.

As used herein, a device function may refer to any operation or function of the aerosol-generating device, which involves operative control of the aerosol-generating device and/or one or more components of the aerosol-generating device by the control circuitry, such as operative control of one or more of the at least one heating element, the energy storage, and one or more user interfaces of the aerosol-generating device.

In an example, the one or more device functions can include a main heating function of the aerosol-generating device, which is also referred to herein as main device function. The main heating function may refer to operation of the aerosol-generating device to heat at least a part of the at least one heating element, the at least part of the aerosol-generating article and/or the substrate to a temperature at or above a predetermined heating temperature of the heating element, the aerosol-generating article and/or the aerosol-generating substrate to generate aerosol. As used herein the predetermined heating temperature may refer to or denote a temperature, level of temperature or temperature range above room temperature sufficient to generate aerosol and/or release aerosol from the aerosol-generating article or substrate, which released aerosol can be inhaled by the user.

Depending on the type and composition of the specific aerosol-generating article or substrate to be used with the device, the predetermined heating temperature may be in a range between 250 degree Celsius and 450 degree Celsius, particularly between 270 degree Celsius and 430 degree Celsius, more particularly between 315 degree Celsius and 355 degree Celsius. These temperatures may be suitable operating or heating temperatures sufficient to allow volatile compounds to be released from the aerosol-generating substrate.

The main heating function of the aerosol-generating device may be executed or performed during or in a usage session. As used herein, a usage session may refer to a period of time, during which a user may use the device to generate, consume, experience or inhale aerosol using the aerosol-generating device. Therein, a usage session may be finite. In other words, a usage session may have a start, an end and a duration. The duration of the usage session as measured by time may be influenced by use during the usage session. The duration of the usage session may have a maximum duration determined by a maximum time from the start of the usage session. The duration of the usage session may be less than the maximum time if one or more monitored parameters reaches a predetermined threshold before the maximum time from the start of the usage session. By way of example, the one or more monitored parameters may comprise one or more of: i) a cumulative puff count of a series of puffs drawn by a user since the start of the usage session, and ii) a cumulative volume of aerosol evolved from the aerosol-forming substrate since the start of the usage session.

It is noted that operation of the aerosol-generating device in a usage session to generate aerosol may be synonymously used herein with operating the aerosol-generating device in the main device function, with operating the device to perform the main heating function, with executing or performing the main device heating function and/or with heating one or more of the heating element, the aerosol-generating article and the aerosol-generating substrate to a temperature at or above the predetermined heating temperature.

The one or more device functions can include one or more auxiliary device functions. Therein, an auxiliary device function may refer to an operation or function of the aerosol-generating device, which is different than the main heating function of the aerosol-generating device. For example, an auxiliary device function may differ from the main heating function in terms of one or more of a type of the function, a purpose of the function, a duration of the function, one or more components involved in performing the function, and energy consumption. However, an auxiliary device function can differ in any other aspect, characteristic or element with respect to the main heating function.

As used herein, an evaluation of the determined storage status with respect to the at least one threshold value associated with at least one device function of the aerosol-generating device may include analysing the determined storage status with respect to the at least one threshold value. For example, the at least one threshold value may be associated with the at least one device function based on, by defining and/or by encoding one or more criteria that should preferably be fulfilled, such that the at least one device function can be executed or performed by the aerosol-generating device. Accordingly, by evaluating the storage status against the threshold value, the storage status can be analysed in terms of the one or more criteria that should preferably be fulfilled, to perform the at least one device function.

Optionally, such analysis or evaluation of the storage status may include a comparison of the storage status to the at least one threshold value. Alternatively or additionally, the analysis or evaluation of the storage status may include converting the storage status and/or the threshold value into a converted storage status and/or converted threshold value. In a non-limiting example, a threshold value or storage status related to electrical energy may be converted to a threshold value or storage status related to capacity, and vice versa.

Optionally, one or more storage status variables may be derived from or computed based on the determined storage status for evaluating the storage status, for example based on comparing the one or more derived storage status variables to the at least one threshold value. Alternatively or additionally, one or more further threshold values may be provided, computed or derived from the at least one threshold value for evaluating the storage status. One or more of the threshold values or further threshold values may, for example, be intercompared to the storage status and/or one or more storage status variables derived therefrom.

As noted above, the at least one threshold value may be associated with or related to at least one device function. For instance, the threshold value may be associated with heating of the aerosol-generating article, substrate and/or heating element, for example in one or more usage sessions or in one or more pause modes, as discussed in more detail hereinbelow.

In a non-limiting example, the at least one threshold value may be descriptive and/or indicative of one or more criteria, predefined criteria, requirements and/or prerequisites for performing one or more device functions associated with the at least one threshold value. In other words, the at least one threshold value may reflect or encode one or more criteria, requirements and/or prerequisites that should be fulfilled in order to perform the at least one device function and/or in order to enable the at least one device function, for example to allow execution of the at least one device function by the user.

In an example, the at least one threshold value may be indicative of and/or correlate with an energy consumption of the aerosol-generating device for performing or executing one or more device functions. For instance, the at least one threshold value may be indicative of and/or correlate with an amount of energy required for performing one or more device functions. Alternatively or additionally, the at least one threshold value may be associated with an energy consumption for performing the one or more device functions.

In an example, the at least one threshold value may correlate with and/or be indicative of an amount of energy required for performing a main heating function of the aerosol-generating device in one or more usage sessions. Alternatively or additionally, the at least one threshold value may correlate with and/or be indicative of an amount of energy required for heating the at least part of the aerosol-generating article to or above the predetermined heating temperature during or in one or more usage sessions. Alternatively or additionally, the at least one threshold value may correlate with and/or be indicative of an amount of energy required for performing one or more auxiliary device functions.

Alternatively or additionally, the at least one threshold value associated with the at least one device function may be associated with and/or indicative of one or more characteristics of the energy storage. For instance, the at least one threshold value may be indicative of one or more predefined criteria the energy storage should fulfil in order to enable the at least one device function.

In an example, the determined storage status may include at least one of a current energy level of electrical energy stored in the energy storage, and a health status of the energy storage indicative of an amount of electrical energy currently storable in the energy storage. For example, the current energy level may refer to or be indicative of a current state of charge of the energy storage. The state of charge may also be referred to herein as charging state. Further, the energy storable in the energy storage may correlate with or be indicated by a current capacity or storage capacity of the energy storage. Accordingly, the storage status may be indicative of one or both a current state of charge of the energy storage and a storage capacity of the energy storage.

Optionally, the storage status may include one or more storage variables or storage status variables, for example one indicative of the current state of charge of the energy storage and a further one indicative of the storage capacity of the energy storage.

In particular when the aerosol-generating device is frequently used or operated to generate aerosol, a quality or health of the energy storage, which may relate to or be indicative of its capability to store electrical energy, may decrease over time. This effect may also be referred to as aging or aging effect of the energy storage. Based on evaluating the storage status, the aging effect of the energy storage can be comprehensively considered for energy management, which may allow to further increase overall energy efficiency. Also, taking the aging effect into consideration may allow to dynamically adjust the repertoire of device functions available to the user in accordance with the energy storage's health state or status.

Further, limitations in capacity of the energy storage and/or frequent use of the device to generate aerosol in one or more usage sessions may result in frequent charging and discharging of the energy storage. Potentially, such frequent charging and discharging of the energy storage may adversely affect a quality of the energy storage and its capability to store electrical energy may potentially decrease over time. After a certain lifetime or service-life of the energy storage, the aerosol-generating device or at least the energy storage may be replaced. Based on evaluating the storage status, also excessive charging and/or discharging may be avoided or reduced, which may increase the lifetime of the energy storage and the aerosol-generating device.

It is noted that in the context of the present disclosure the terms energy and capacity of the energy storage can be interchangeably used and/or can be converted into one another. For instance, an energy value, for example given in units of Wh or mWh, may be computed based on the capacity of the energy storage, for example given in Ah or mAh, multiplied by a nominal voltage of the energy storage.

According to an example, evaluating the determined storage status may include comparing the determined storage status to the at least one threshold value. Optionally, the at least one threshold value may be indicative of a threshold storage status. For example, the at least one threshold value may be indicative of a threshold or minimum amount of electrical energy currently stored in the energy storage. Alternatively or additionally, the at least one threshold value may be indicative of a threshold or minimum state of charge of the energy storage.

For instance, upon determining by the control circuitry that the storage status reaches or exceeds the at least one threshold value, the control circuitry may enable the at least one device function for execution, for example by the user. Alternatively or additionally, upon determining that the storage status is below the at least one threshold value, the control circuitry may disable the at least one device function for execution, for example by the user.

Alternatively, upon determining by the control circuitry that the storage status reaches or exceeds the at least one threshold value, the control circuitry may disable the at least one device function for execution, for example by the user. Alternatively or additionally, upon determining that the storage status is below the at least one threshold value, the control circuitry may enable the at least one device function for execution, for example by the user.

In an example, the at least one threshold value may be indicative of a threshold energy or energy amount required for operating the aerosol-generating device during at least one usage session to generate inhalable aerosol based on heating the aerosol-generating article, in particular to a temperature at or above a predetermined heating temperature to generate aerosol. Alternatively or additionally, the at least one threshold value may be indicative of a threshold energy or energy amount required for performing the main heating function of the aerosol-generating device. Based on evaluating the storage status with such threshold energy, it may be reliably determined whether or not the amount of energy currently stored in the energy storage is sufficient to complete the at least one usage session and/or is sufficient to allow the user to experience or consume at least one aerosol-generating article in one or more usage sessions.

For instance, the storage status may be indicative of a current state of charge and the control circuitry may be configured to compare the storage status with the threshold value that may be indicative of a threshold energy or energy amount required for operating the aerosol-generating device during at least one usage session to generate inhalable aerosol based on heating the aerosol-generating article, in particular to a temperature at or above a predetermined heating temperature to generate aerosol. Upon determining that the storage status or state of charge indicated by the storage status reaches or exceeds the threshold value, the at least one device function, for instance the main heating function, may be enabled. Alternatively or additionally, upon determining that the storage status or state of charge indicated by the storage status is below the threshold value, the at least one device function, for instance the main heating function, may be disabled. Accordingly, use of the aerosol-generating device by a user to generate aerosol in one or more usage sessions may be prevented in the latter case.

In an exemplary implementation, the control circuitry may be configured to enable the at least one device function upon determining, based on the evaluation, that a current energy level of electrical energy stored and/or storable in the energy storage is sufficient for operating the aerosol-generating device during at least one usage session to generate inhalable aerosol based on heating the aerosol-generating article to a temperature at or above a predetermined heating temperature to generate aerosol. Accordingly, the control circuitry may be configured to determine, based on the evaluation, whether the amount of energy currently stored in the energy storage is sufficient to complete one or more usage sessions.

In a further example, the at least one threshold value may be indicative of a threshold energy required for performing a main heating function of the aerosol-generating device during at least one usage session, the main heating function involving supplying electrical energy to the at least one heating element to heat at least a part of the aerosol-generating article to a temperature at or above a predetermined heating temperature to generate aerosol. Alternatively or additionally, the control circuitry may be configured to enable a main heating function upon determining, based on the evaluation, that a current energy level of electrical energy stored and/or storable in the energy storage is sufficient for performing the main heating function during at least one usage session to generate inhalable aerosol. Accordingly, the main heating function may only be enabled by the control circuitry if the energy stored in and/or storable in the energy storage is sufficient to execute the main function at least one time during at least one usage session, for example using at least one aerosol-generating article.

Optionally, in response to determining that the energy stored in the energy storage is sufficient to perform the main heating function in one or more usage sessions, for example using one or more aerosol-generating articles, the control circuitry may indicate availability of one or more usage sessions to the user, for example based on actuating one or more user interfaces of the aerosol-generating device, such as a control light, one or more LEDs or a display.

Further optionally, the control circuitry may be configured to determine, based on the evaluation, a number of usage sessions and/or a number of times the main function can be executed before re-charging the energy storage, respectively with a fully charged energy storage. Optionally, the number of usage sessions or experiences of aerosol-generating articles available to the user can be indicated and/or notified to the user, for example based on actuating one or more user interfaces of the aerosol-generating device.

In an exemplary implementation, the at least one threshold value may be indicative of a threshold energy required for completing a current usage session to generate inhalable aerosol based on heating the aerosol-generating article, in particular to a temperature at or above a predetermined heating temperature to generate aerosol. Therein the current usage session may refer to a currently ongoing usage session or usage session that has already been started or initiated by the user. Accordingly, the control circuitry may be configured to determine during an ongoing usage session, whether sufficient energy is stored in the energy storage to complete the usage session. Whether or not the current usage may be completed may optionally be indicated and/or notified by the control circuitry to the user, for example based on actuating one or more user interfaces of the device.

Alternatively or additionally, the at least one threshold value may be indicative of a threshold energy required for completing a predefined number of usage sessions to generate inhalable aerosol based on heating the aerosol-generating article to a temperature at or above a predetermined heating temperature to generate aerosol. The predefined number of usage sessions may be indicative of a maximum number of usage sessions completable with the amount of energy currently stored in the energy storage, for example before re-charging the energy storage and/or after the energy storage has been charged. It is noted that after the energy storage has been charged may refer to or may be synonymously used herein with “after the energy storage has been charged the last time” or “between two consecutive charging events”. Alternatively or additionally, the predefined number of usage sessions may be indicative of a number of usage sessions completable with the device or available to the user with a complete charge of the energy storage and/or with a fully charged energy storage. Alternatively or additionally, the pre-defined number of usage sessions may be indicative, descriptive and/or representative of a number of usage sessions the aerosol-generating device is operable or can be operated before a next, subsequent or consecutive charging event. Alternatively or additionally, the pre-defined number of usage sessions may be indicative, descriptive and/or representative of a number of usage sessions the aerosol-generating device is operable or can be operated with a charged or fully charged energy storage, and/or between two temporally consecutive charging events.

It is noted that a complete charge of the energy storage or a fully charged energy storage may refer to or denote an energy storage that has been charged up to a maximum energy capacity, such that a maximum amount of electrical energy storage is stored in the energy storage. Therein, the maximum energy capacity, also referred to herein as storage capacity or capacity of the energy storage, may be defined by physical or chemical limits of the energy storage. Alternatively or additionally, the maximum energy capacity may be defined by a corresponding configuration of the energy storage.

Optionally, the pre-determined number of usage sessions may be indicated and/or notified to the user, for example based on actuating one or more user interfaces by the control circuitry.

For example, the predetermined number of usage sessions may be at least one usage session, preferably at least two usage sessions, at least three usage sessions, at least four usage sessions or even more usage sessions. In turn, a minimum number of one, two, three, four or even more aerosol-generating articles may be used by the user to generate aerosol in at least one, two, three, four or even more usage sessions.

In an example, the control circuitry may be configured to enable the at least one device function, for example the main heating function and/or one or more auxiliary device functions, upon determining, based on the evaluation, that a current energy level of electrical energy stored and/or storable in the energy storage is sufficient for completing a current usage session and/or for completing a predefined number of usage sessions to generate inhalable aerosol based on heating the aerosol-generating article to a temperature at or above a predetermined heating temperature to generate aerosol. Alternatively or additionally, the control circuitry may be configured to disable the at least one device function, for example the main heating function or one or more auxiliary device functions, upon determining, based on the evaluation, that a current energy level of electrical energy stored and/or storable in the energy storage is insufficient for completing a current usage session and/or for completing a predefined number of usage sessions to generate inhalable aerosol based on heating the aerosol-generating article to a temperature at or above a predetermined heating temperature to generate aerosol.

The aerosol-generating device may comprise a heating circuitry and/or a heating arrangement that may be operated in at least two operation modes, an aerosol-releasing mode and a pause mode. Accordingly, the aerosol-generating device may be operated in at least two operation modes. The heating circuitry may be part of or may be provided by the control circuitry. The aerosol-generating device, the heating circuitry and/or the control circuitry may be configured to heat the heating element, the aerosol-generating article and/or the substrate at a first temperature level in the aerosol-releasing mode. Therein, the first temperature level may correspond to the predetermined heating temperature or a temperature above. The aerosol-generating device may further be configured to heat the heating element, the aerosol-generating article and/or the substrate at a second temperature level below the first temperature level in a pause mode of the aerosol-generating device. The second temperature level may, for example, refer to a temperature above room temperature and below the first temperature level.

In yet another example, the at least one threshold value may be indicative of a threshold energy required for operating the aerosol-generating device during at least one usage pause in a pause mode interrupting a usage session.

A usage session or user experience, also referred to as experience of an aerosol-generating article herein, may be interrupted, for example by switching the device into the pause mode, and resumed by a user at a later, wherein the aerosol-generating article or substrate is kept in pause mode of the aerosol-generating device at a temperature below the first temperature level and/or below the predetermined heating temperature used during normal use of the device (in particular during a user experience or usage session), but still above or well above room temperature. That is, the second temperature level preferably may be chosen such as to avoid degradation of the non-depleted substrate or aerosol-generating article. In particular, the second temperature level may be chosen such as to be sufficiently low in order to minimize depletion of the substrate or article during the pause mode, and at the same time to be sufficiently high in order to avoid vapor to condensate in the device which otherwise could affect the quality of the non-depleted aerosol-generating substrate or article.

During use of the device, in particular when a user experience or usage session is to take place, the heating element, the heating circuitry and/or the heating arrangement may be operated in the aerosol-releasing mode, whereas during a use pause of the device, that is, when no user experience or usage session is to take place and/or when a usage session is interrupted by a pause, the aerosol-generating device may be operated in the pause mode. During both, the aerosol-releasing mode and the pause mode of the aerosol-generating device, the heating element, a heating circuitry and/or a heating arrangement may be in operation, in particular in heating operation, yet at different temperature levels, namely, at a first temperature level during the aerosol-releasing mode, which may be chosen to be sufficiently high in order to generate an aerosol, and at a second temperature level below the first temperature level during the pause mode, which may be chosen to be sufficiently low in order to minimize depletion of the substrate, whilst avoiding degradation.

Depending on the type and composition of the specific aerosol-generating article or substrate to be used with the device, the first temperature level may be in a range between 250 degree Celsius and 450 degree Celsius, particularly between 270 degree Celsius and 430 degree Celsius, more particularly between 315 degree Celsius and 355 degree Celsius. These temperatures may be suitable operating or heating temperatures sufficient to allow volatile compounds to be released from the aerosol-generating article or substrate, for example during one or more usage sessions and/or when operating the device in the aerosol releasing mode. For example, the first temperature level and/or heating temperature for liquid aerosol-generating articles or substrates may be lower than the first temperature level for solid aerosol-generating articles or substrates.

In general, the second temperature level may be chosen to maintain a usability of the aerosol-generating article or substrate for a prolonged time. The second temperature level may also depend on the type and composition of the aerosol-generating article or substrate to be used with the device. Accordingly, the second temperature level may be in a range between 175 degree Celsius and 225 degree Celsius, particularly between 185 degree Celsius to 215 degree Celsius, more particularly between 195 degree Celsius and 205 degree Celsius. These temperatures may be sufficiently low in order to minimize depletion of the substrate during the pause mode but at the same time sufficiently high in order to avoid vapor to condensate in the device, which could lead to degradation of the aerosol-generating article or substrate.

In order to avoid condensation effects in the device, in particular to avoid condensation of substances in the aerosol-generating article or substrate, the second temperature level may be at least 150 degree Celsius, in particular at least 175 degree Celsius, preferably at least 185 degree Celsius, more preferably at least 195 degree Celsius.

Vice versa, in order to minimize depletion of the substrate or article during the pause mode the second temperature level may be at most 220 degree Celsius, in particular at most 225 degree Celsius, preferably at most 215 degree Celsius, more preferably at least 205 degree Celsius. In particular, the second temperature level may be chosen such as to reduce the formation of aerosols by at least 50 percent compared to the aerosol-releasing mode.

In relative terms, the second temperature level may be lower than the first temperature level, for example by at least 50 degree Celsius, in particular at least 75 degree Celsius, more particularly at least 100 degree Celsius.

The temperature values given above preferably may be average temperatures of the aerosol-generating article or substrate during operation of the device. In addition, as already mentioned, the temperature values may depend, inter alia, on the type and composition of the aerosol-generating article or substrate to be used with the device.

As used herein, the pause mode may refer to a first operational mode of aerosol-generating device, in which the heating element, the heating circuitry and/or a heating arrangement may be operated during an operation pause, that is, a use pause of the aerosol-generating device, that is, when a user experience or usage session is paused and aerosol generation may not take place, or at least may be reduced to a minimum level. That is, in the pause mode the aerosol-generating device is in a use pause.

Vice versa, the aerosol-releasing mode may refer to a second operational mode of the aerosol-generating device, which is the normal heating operational mode of the heating element, circuitry, and/or arrangement for aerosol generation, in which heating element, the heating circuitry and/or a heating arrangement may be operated during use of the device by a user, that is, when a user experience or usage session takes place, in particular when aerosol generation takes place. In general, aerosol generation may take place continuously or on demand, in particular on a puff basis, that is, on demand of a user when taking a puff.

The aerosol-generating device may optionally comprise at least one sensor configured to output a sensor signal indicative of the device being in operation by a user, that is, in use by a user, or in an operation pause, that is, a use pause. Advantageously, such a sensor may facilitate to automatically detect whether operation of the heating arrangement can be switched into the pause mode since the device is currently not in use and thus in an operation pause that is, a use pause. Thus, aerosol generation may be stopped in a timely manner in order to avoid an ongoing but undesired depletion of the aerosol-forming substrate. Likewise, such a sensor may facilitate to automatically detect whether operation of the heating arrangement is to be switched back into the aerosol-releasing mode, that is, back into the normal heating operational mode for aerosol generation when a user wants to resume a user experience or usage session.

In an example, the control circuitry may be configured to switch and/or configure the aerosol-generating device into the aerosol-releasing mode or the pause mode based on the evaluation of the determined storage status with respect to the at least one threshold value.

For instance, the control circuitry may be configured to enable and/or allow the at least one device function upon determining, based on the evaluation, that a current energy level of electrical energy stored and/or storable in the energy storage is sufficient for operating the aerosol-generating device during at least one usage pause in a pause mode interrupting a usage session.

In an exemplary implementation, the at least one threshold value may be indicative of and/or correlate with a threshold energy required for performing at least one auxiliary device function of the aerosol-generating device. Alternatively or additionally, the control circuitry may be configured to enable and/or allow at least one auxiliary device function upon determining, based on the evaluation, that a current energy level of electrical energy stored and/or storable in the energy storage is sufficient for performing the at least one auxiliary device function of the aerosol-generating device. Accordingly, the storage status may be analysed in terms of whether sufficient energy is stored therein to execute or complete the at least one auxiliary device function. If the energy stored and/or storable in the energy storage is sufficient for completing the auxiliary device function, the function may be enabled or activated. Hence, the repertoire of functions or functionalities available to the user may be expanded or increased. Alternatively or additionally, upon determining that the energy stored and/or storable in the energy storage is not sufficient for performing or completing the auxiliary device function, said function may be prevented, disabled and/or deactivated, for example such that it cannot be initiated by the user and/or such that it is unavailable to the user.

In an example, the auxiliary device function may be non-related to and/or differ from heating the aerosol-generating device during a usage session to generate aerosol, for example based on heating the aerosol-generating article to a temperature at or above a predetermined heating temperature to generate aerosol. Alternatively or additionally, the auxiliary device function may be associated with a pause mode interrupting a usage session.

In yet another example, the at least one device function can include at least one of a main heating function of the aerosol-generating device to generate aerosol in at least one usage session based on heating the aerosol-generating article to a temperature at or above a predetermined heating temperature to generate aerosol, and at least one auxiliary device function of the aerosol-generating device, which differs from the main heating function. In other words, the at least one device function, which the at least one threshold value may be associated with and/or which may be enabled or disabled by the control circuitry based on the evaluation of the storage status, may refer to or include one or both the main heating function and an auxiliary device function that differs from the main device function. For instance, the control circuitry may evaluate the storage status at least in terms of the main heating function, and optionally in terms of one or more auxiliary device functions.

For example, the auxiliary device function may include one or more of operating the aerosol-generating device at reduced energy consumption with respect to operation during a usage session or in the aerosol-releasing mode to generate aerosol, heating the at least one heating element and/or the aerosol-generating article to a temperature below a heating temperature sufficient to generate aerosol, heating the at least one heating element and/or the aerosol-generating article to a temperature above room temperature and below a heating temperature sufficient to generate aerosol, heating the at least one heating element and/or the aerosol-generating article to a temperature above room temperature and below a heating temperature sufficient to generate aerosol for a predefined period of time, for example during a use pause, activating or deactivating haptic control of the aerosol-generating device, activating or deactivating one or more user interfaces of the aerosol-generating device, operating a user interface of the aerosol-generating device, operating a communication circuitry of the aerosol-generating device for communicatively coupling the aerosol-generating device to a computing device or receiving device, operating a sensor of the aerosol-generating device, and operating a biometric sensor of the aerosol-generating device for user authentication. Therein, operating may include one or more of actuating, enabling, disabling, activating and deactivating. Generally, any device function other than the main heating function may be regarded or referred to as auxiliary device function in the context of the present disclosure.

In an exemplary implementation, the at least one threshold value may correlate with a threshold energy required for a) performing a main heating function of the aerosol-generating device in at least one usage session to generate aerosol, and b) for performing an auxiliary device function different than, other than and/or non-related to the main heating function of the aerosol-generating device. In other words, the at least one threshold value may be indicative of an energy consumption when performing both the main heating function and the at least one auxiliary device function. It should be noted that also two threshold values may be used, a first threshold value indicative of the energy required for performing the main heating function of the aerosol-generating device in at least one usage session to generate aerosol, and a second threshold value indicative of the energy required for performing the auxiliary device function.

Optionally, the at least one threshold value may correlate with a threshold energy required for operating the aerosol-generating device a) during at least one usage session in an aerosol-releasing mode generating inhalable aerosol based on heating the aerosol-generating article to a temperature at or above the predetermined heating temperature, and b) during a usage pause in a pause mode interrupting the at least one usage session. Alternatively or additionally, the control circuitry may be configured to enable a) a main heating function of the aerosol-generating device in at least one usage session to generate aerosol based on heating the heating element, the article and/or substrate to a temperature at or above the predetermined heating temperature, and b) at least one auxiliary device function of the aerosol-generating device different than, other than, and/or non-related to the main heating function, upon determining, based on the evaluation, that a current energy level of electrical energy stored and/or storable in the energy storage is sufficient for performing the main heating function and the at least one auxiliary device function of the aerosol-generating device. Alternatively or additionally, upon determining based on the evaluation, that a current energy level of electrical energy stored and/or storable in the energy storage is insufficient for performing the main heating function and the at least one auxiliary device function of the aerosol-generating device, the control circuitry may disable or prevent one or both the main heating function and the at least one auxiliary device function.

For instance, the at least one auxiliary device function may refer to and/or be associated with heating of the heating element, the aerosol-generating article and/or the substrate to the second temperature level or second temperature during the use pause or pause mode of the device, wherein the second temperature level may be above room temperature and below the predetermined heating temperature or first temperature level. Accordingly, the control circuitry may reliably detect whether the energy storage stores sufficient electrical energy to perform the main heating function in one or more usage sessions and to perform at least one use pause interrupting the one or more usage sessions. Generally, this may allow to reliably determine whether the device can be operated in one or more use pauses. Also, operation of the device in the pause mode may dynamically be allowed or prevented, depending on the storage status of the energy storage. In turn, the number of usage sessions may be maximized for the user, for example based on preventing the pause mode and/or the at least one auxiliary device function.

In an exemplary implementation, the control circuitry may be configured to trigger or request re-charging of the energy storage based on the evaluation of the determined storage status with respect to the at least one threshold value. For instance, upon determining that an insufficient amount of energy is stored and/or storable in the energy storage, the control circuitry may re-quest the user to re-charge the energy storage, for example to a predetermined state of charge or to a maximum state of charge of the energy storage. Accordingly, triggering recharging of the energy storage may include indicating to the user, for example based on actuation of one or more user interfaces, that the device should be re-charged. Alternatively or additionally, triggering recharging can include rendering the device inactive for further use.

In an example, the at least one threshold value may be indicative of a fraction of a current maximum capacity of the energy storage, in particular about 70% to about 100% of the current maximum capacity, preferably at least about 90% of the current maximum capacity. As noted above, alternatively or in addition to capacity threshold, one or more corresponding energy thresholds may be used.

For instance, the main heating function and optionally one or more auxiliary device functions may be enabled upon determining by the control circuitry that the current capacity of the energy storage, state of charge and/or the amount of energy stored therein is or corresponds to at least 70%, at least 80% or at least 90% of the current maximum capacity of the energy storage. Therein, the current maximum capacity of the energy storage may be indicative of the maximum amount of electrical energy currently storable in the energy storage. For example due to aging effects of the energy storage, the current maximum capacity may be equal to or below an initial or nominal capacity of the energy storage. The latter may, for example, refer to the capacity of the storage without aging effect.

In an example, determining the storage status may include determining a current energy level of electrical energy stored and/or storable in the energy storage relative to a current maximum capacity of the energy. Therein, the current energy level of electrical energy stored may be indicative of the current state of charge of the energy storage. Alternatively or additionally, the current energy level of electrical energy storable may be indicative of the current capacity, storage capacity and/or current maximum capacity of the energy storage.

The control circuitry may be configured to determine a current maximum capacity of the energy storage. Alternatively or additionally, the control circuitry may be configured to determine an initial maximum capacity and/or initial storage capacity of the energy storage. Optionally, the control circuitry may be configured to determine a relative percentage or fraction of the current maximum capacity with respect to the initial maximum capacity of the energy storage.

In an example, the at least one threshold value may be indicative of a fraction of an initial maximum capacity of the energy storage, in particular between about 35% and about 75% of the initial maximum capacity. For instance, if the control circuitry determines that the current maximum capacity or current storage capacity of the energy storage falls below the fraction of the initial maximum capacity or initial storage capacity indicated by the threshold value, one or both the main heating function and at least one auxiliary device function, for example the pause mode, may be disabled.

Alternatively or additionally, the at least one threshold value may be indicative of whether a predefined charging stage the energy storage has been reached in a previous charging event, in particular a constant voltage stage of about 3 to 5 V, for example about 3.4 V to about 3.9 V, in particular about 3.65 V charging voltage. Generally, this may allow to determine the amount of electrical energy currently stored in the energy storage.

Optionally, the control circuitry may be configured to determine whether the energy storage has been charged to a predefined charging stage in a previous charging event. As noted above, the predefined charging stage may relate to a constant voltage stage of about 3 to 5 V, for example about 3.4 V to about 3.9 V, in particular about 3.65 V charging voltage. Based on determining the charging stage reached in the previous charging event, the control circuitry may determine the amount of electrical energy currently stored in the energy storage, and hence available for one or more device functions.

It should be noted that any other characteristic of the previous charging event may be used to estimate the amount of energy stored in the energy storage. For instance, a charging current profile (or charging current as a function of charging time) may be used. Alternatively or additionally, a charging time may be used. For instance, the control circuitry may be configured to determine whether the energy storage has been charged by a predefined charging time, according to a predefined charging profile and/or to a predetermined charging current stage in the previous charging event. As used herein a charging event may refer to the time period the energy storage is supplied with electrical energy from an energy supply and/or from a companion device of the aerosol-generating system.

In an exemplary implementation, determining the storage status may include determining a health status of the energy storage. Alternatively or additionally, the control circuitry may be configured to determine a health status of the energy storage. Alternatively or additionally, determining the storage status may include determining an age of the energy storage

As used herein, a health status of the energy storage may be indicative of and/or correlate with a maximum energy currently storable in the energy storage, with a current maximum capacity, and/or current storage capacity of the energy storage. For example due to frequent charging and discharging of the energy storage, the health of the energy storage may degrade over time, which is also referred to as age effect or aging of the energy storage. Accordingly, determining the health or health status of the energy storage may include determining the current or an actual maximum capacity of the energy storage. Determining the health status of the energy storage may generally allow for an energy management that takes aging of the energy storage into consideration, thereby allowing for a more precise and more efficient energy management. Also, usability of the device for the user may be improved, for example in terms of maximizing the number of usage sessions the device can be used by the user before re-charging the energy storage and/or with a fully charged energy storage.

In an exemplary configuration, the at least one threshold value may be indicative of a threshold health status and/or threshold age of the energy storage. Accordingly, evaluating the storage status with respect to the threshold value may involve analysing the energy storage in terms of its health status. Further, the at least one device function may be enabled or disabled depending on the determined and evaluated health status.

In an example, the control circuitry may be configured to determine a health status of the energy storage based on determining one or more of a current maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated or used to generate aerosol, charging data related to charging of the energy storage in one or more charging cycles, and a total energy consumption of the aerosol-generating device. Alternatively or additionally, the control circuitry may be configured to determine and/or monitor one or more of a current maximum capacity of the energy storage, an initial maximum capacity of the energy storage, a current maximum capacity of the energy storage with respect to an initial maximum capacity of the energy storage, a number of usage sessions the aerosol-generating device has been used to generate aerosol, for example since the energy storage has been charged the last time, an operation time the aerosol-generating device has been operated to generate aerosol, charging data related to charging of the energy storage in one or more charging cycles, and a total energy consumption of the aerosol-generating device. Any one or more of these quantities may be used to reliably determine, estimate and/or compute the health status.

In yet another exemplary implementation, determining the storage status may include determining a current maximum capacity and/or current storage capacity of the energy storage indicative of a maximum amount of energy storable in the energy storage and determining a current energy level or amount of electrical energy currently stored in the energy storage.

Optionally, the at least one threshold value may include a first threshold value for a current energy level, state of charge and/or amount of energy available in the energy storage, and a second threshold value for a current maximum capacity of the energy storage indicative of a maximum amount of energy currently storable in the energy storage, wherein the control circuitry may be configured to compare a determined current energy level to the first threshold value and to compare a determined current maximum capacity of the energy storage to the second threshold value.

Taking the storage capacity or the amount of energy theoretically storable, for example theoretically storable, in the energy storage as well as the actual state of charge of the energy storage into account, can allow for a comprehensive energy management. In turn, the repertoire of functions of the device can be tailored to user-specific demands. For example, the number of usage sessions or experiences can be maximized based on preventing one or more auxiliary device functions or limited based on preventing the main heating function.

In yet another exemplary implementation, the at least one threshold value includes a first threshold value for a current energy level or amount of energy available in the energy storage, and a second threshold value for a current maximum capacity of the energy storage indicative of a maximum amount of energy currently storable in the energy storage, wherein the control circuitry may be configured to compare a determined current energy level to the first threshold value and to compare a determined current maximum capacity of the energy storage to the second threshold value.

In yet another exemplary implementation, the at least one threshold value may be adjustable and/or variable. Alternatively or additionally, the control circuitry may be configured to adjust, change, replace, modify, alter and/or vary the at least one threshold value.

For example, the at least one threshold value may be adjusted by the control circuitry based on or in accordance with user-specific needs or demands. For instance, the number of usage sessions and/or use pauses available to or initiable by the user may be optimized, for example maximized. Alternatively or additionally, the number of usage sessions may be defined by the user. Hence, versatility and flexibility of the device can be increased.

In an example, the control circuitry may be configured to adjust, adapt and/or vary the at least one threshold value based on historic data indicative of a past or previous usage of the aerosol-generating device and/or indicative of a consumption behaviour of a user. In an example, the control circuitry may be configured to determine and/or derive the consumption behaviour of the user from the historic data.

It has been found that different users may have a different consumption behaviour, which may for example refer to or be defined as consumption time for and/or duration of one or more usage sessions, during which the heating element, the aerosol-generating article and/or the substrate may be heated to a temperature at or above the predetermined heating temperature. Therefore, the energy needed or consumed by a user per usage session may vary from user to user. Such different consumption behaviour may be reflected in the adjustable threshold value, and hence an energy management can be tailored in accordance with the consumption behaviour of a particular user.

Exemplary users may consume, for example, less than about 100 mAh, in particular less than about 95 mAh, for example about 60 mAh to about 85 mAh, energy per usage session and/or per aerosol-generating article. In case a fixed threshold value for the maximum amount of energy stored and/or storable in the energy storage were used, such users would have to wait for a longer charging time than what is actually needed to experience the next usage session. In other words, some users, in particular those who have a consumption behaviour that requires less energy per usage session, could be forced to re-charge the energy storage when a fixed threshold value was used, although the energy storage may still store enough energy to complete one or more usage sessions. Such scenarios can be effectively prevented or avoided based on adjusting the threshold value. In other words, an adjustable or variable threshold value may allow to flexibly cope with different needs from different users.

Furthermore, when using a fixed threshold value, a user may not be allowed to pause between puffs and/or during a usage session. By adjusting the threshold value, the device may be flexibly controlled by the user, for example between puffs and/or during a usage session. Overall, functionality of the device and user experience can be improved.

The aerosol-generating device may optionally include a data storage or memory. For example, the historic data may be stored in the data storage. Alternatively or additionally, the historic data may be stored at a computing device, for example a server, a server network or mobile device, communicatively couplable to the aerosol-generating device, for example by means of a communication circuitry of the aerosol-generating device.

As used herein, the computing device may refer to any device or apparatus with data processing capabilities, such as a server, a server network, a mobile device, a smart phone, a tablet, a smart device, and a smart wearable. The computing device may be configured to communicate or transmit the data, for example wirelessly or by wire, to the aerosol-generating device. Accordingly, the computing device may include a communication interface or circuitry configured to transmit data, for example the historic data or other data, to the aerosol-generating device.

Generally, the historic data may be indicative of a consumption behaviour of the user in one or more usage sessions. In particular, data related to one or more usage sessions may be stored in the historic data. For example, one or more of start times, stop times, durations, energy consumption, a number of puffs in a single usage sessions, a total number of puffs, a number of experiences, a number of usage sessions, a number of aerosol-generating articles used, and other information related to one or more usage sessions can be stored in the historic data.

Alternatively or additionally, data related to or indicative of one or more usage pauses and/or operation of the device in the pause mode may be comprised in the historic data. For instance, start times, stop times and/or durations of one or more usage pauses may be stored in the historic data. Alternatively or additionally, energy consumption during one or more usage pauses may be stored in the historic data.

Alternatively or additionally, the historic data may comprise data related to and/or indicative of an energy consumed for performing one or more device functions.

In an example, the control circuitry may be configured to compute, based on historic data indicative of an operation of the aerosol-generating device to generate aerosol, an amount of energy used in a single usage session, and to adjust the at least one threshold value based on the determined amount of energy used in the single usage session. Optionally, the determined amount of energy used in a single usage session may correspond to and/or be used as the at least one threshold value. Generally, the energy used in a single usage session may refer to the energy actually used in a real usage session. Alternatively or additionally, the energy used in a single usage session may refer to or denote a mean or average of energy used or consumed per usage session. It is noted that the energy used in a single usage session may substantially correspond to the energy required for performing the main heating function of the aerosol-generating device in a single usage session.

In yet another example, the at least one threshold value may be indicative of a threshold energy or threshold energy value required for operating the aerosol-generating device during at least one usage session to generate inhalable aerosol based on heating the heating element, the aerosol-generating article and/or the substrate to a temperature at or above the predetermined heating temperature, preferably wherein the at least one threshold value may be less than about 100 mAh, for example less than about 95 mAh, preferably between about 60 mAh and 85 mAh. Typical devices currently in use may have a fixed threshold value of about 100 mAh or even more. Hence by reducing the threshold value, more usage sessions or experiences may be provided to the user before re-charging the energy storage and/or with a fully charged energy storage.

In yet another example, the control circuitry may be configured to determine, based on historic data indicative of an operation of the aerosol-generating device to generate aerosol, a maximum number of usage sessions the aerosol-generating device is usable or can be used with a fully charged energy storage, per charge of the energy storage, since the energy storage has been charged the last time and/or between two consecutive charging events. Further, the control circuitry may be configured to adjust the at least one threshold value based on the determined maximum number of usage sessions. Accordingly, the control circuitry may analyse the historic data and compute and/or estimate the maximum number of usage sessions.

Optionally, the maximum number of usage sessions may be indicated and/or notified to the user, for example based on actuating, operating and/or controlling one or more user interfaces. Alternatively or additionally, the control circuitry may be configured to indicate the determined maximum number of usage sessions to the user based on controlling a user interface of the aerosol-generating device. For instance, a number of Light Emitting Diodes, LEDs, may be switched on or off in accordance with the maximum number of usage sessions or other means for notification may be used instead or in addition.

Alternatively or additionally, a currently available number of usage sessions, for example depending on the amount of energy currently stored in the energy storage, may be indicated and/or notified to the user, for example based on actuating one or more user interfaces.

In a further example, the control circuitry may be configured to determine a maximum number of pause modes or usage pauses that can be activated by the user with a fully charged energy storage, after the energy storage shas been charged the last time, per charge of the energy storage, between two consecutive charging events and/or before re-charging the energy storage. Optionally, also the number of pause modes or usage pauses may be indicated and/or notified to the user, for example based on actuating one or more user interfaces.

In yet another exemplary implementation, the control circuitry may be configured to enable and/or allow use of the aerosol-generating device for a usage session to generate aerosol based on heating the heating element, the aerosol-generating article and/or the substrate to a temperature at or above the predetermined heating temperature, for example a subsequent usage session, independent of a number of previous usage sessions, the aerosol-generating device has been used since a previous or the last charging process. Accordingly, regardless of whether the device was used after the last or previous charging process in one or more usage sessions, the control circuitry may enable or disable the at least one device function, in particular the main heating function. Hence, a user-specific energy management can be implemented in the device.

Alternatively or additionally, the at least one threshold value may be adjustable and/or variable based on a user input. Alternatively or additionally, the at least one threshold value may be adjusted and/or varied based on a user input. Alternatively or additionally, the control circuitry may be configured to alter and/or modify the at least one threshold value based on a user input. Such user input may for example be received via one or more user interfaces of the aerosol-generating device. Alternatively or additionally, a user input may be received from a companion device of an aerosol-generating system and/or from a computing device, for example a server or mobile device, communicatively coupled to the aerosol-generating device. Accordingly, the threshold value may be modified by the user according to its preferences or demands. Also, a user may be enabled to adjust its consumption behaviour, for example by limiting or reducing the number of usage sessions per fully charged energy storage.

In an example, the at least one threshold value may be adjustable or adjusted based on a user input indicative of a number of experiences per charging cycle, per charge or per complete charge of the energy storage and/or indicative of a duration of a single usage session. As used herein, a charging cycle may refer to a cycle of fully charging the energy storage and fully discharging the energy storage. Accordingly, the at least one threshold value may be adjustable or adjusted based on a user input indicative of a number of experiences or usage sessions per fully charged energy storage and/or indicative of a duration of a single usage session. Accordingly, the user may modify or adjust the threshold value according to its preferences or demands based on providing a user-intuitive input or information.

For example, a user may specify the duration of a single usage session and/or and the number of usage sessions per fully charged energy storage or per complete charge of the energy storage via one or more user inputs, and the control circuitry may compute a corresponding threshold energy or capacity value. Based on evaluating the determined storage status with the computed threshold energy or capacity value, the number of usage sessions per charging cycle and/or usage sessions with the user-defined duration can be granted to the user.

Alternatively or additionally, a companion device and/or a computing device communicatively couplable to the aerosol-generating device may compute the one or more threshold values based on the user input, and transmit a corresponding control signal indicative of the one or more threshold values to the aerosol-generating device.

It is noted that a fully charged energy storage may refer to charging the energy storage to its current maximum capacity or state of charge, which may be defined by physical or chemical limitations of the energy storage and/or by the control circuitry, for example by configuring a controller of the energy storage to limit the current maximum capacity or state of charge to a particular value. Alternatively or additionally, a fully discharged energy storage may refer to an empty energy storage, current minimum capacity, or minimum state of charge, which may be defined by physical or chemical limitations of the energy storage and/or by the control circuitry, for example by configuring a controller of the energy storage to limit the current minimum capacity or state of charge to a particular value. Upon reaching the minimum capacity, the control circuitry may request, trigger and/or enforce re-charging of the energy storage.

In an exemplary implementation, the control circuitry may be configured to compute and/or derive the at least one threshold value based on historic data collected in one or more past or previous usage sessions. For instance, the aerosol-generating device or control circuitry may be configured to store or record data related to a usage session during operation. For example, one or more of start time, stop time, duration, energy consumption, reduction in amount of energy stored in the energy storage, reduction in capacity of the energy storage, temperature, temperature profile, an indication of an interruption of a usage session by a pause mode, a number of pause modes or usage pauses in a usage session or other information, for example information related to charging of the energy storage, may be stored or recorded by the control circuitry for one or more past or previous usage sessions. The historic data may for example be stored at a data storage of the aerosol-generating device. Alternatively or additionally, the historic data may be transmitted to one or more of a companion device and a computing device, for example a server or mobile device, communicatively couplable to the aerosol-generating device. One or more data elements related to one or more usage sessions and/or one or more usage pauses or pause modes may be contained in the historic data.

The control circuitry may further be configured to process and/or analyse one or more of the data elements of the historic data and determine one or more threshold values for evaluation with the storage status of the energy storage. For instance, one or more threshold values related to an energy consumption per usage session and/or a change in capacity per usage session may be determined by the control circuitry. Hence, the consumption behaviour of the user, as reflected by the historic data, may be used to dynamically adjust the at least one threshold value and/or to adapt a number of usage sessions granted to the user per fully charged energy storage.

In an example, the control circuitry may be configured to determine or compute the at least one threshold value based on a predefined number of previous or past usage sessions. For example, a number of data elements of the historic data corresponding to the predefined number of previous or past usage sessions may be processed and/or analysed by the control circuitry to derive or compute the at least one threshold value for evaluation with the storage status of the energy storage.

In a non-limiting example, only the last usage session or corresponding data element of the historic data may be used to compute the at least one threshold value. Alternatively, two or more, for example between two and 20, preferably between two and 16 previous or past usage sessions or corresponding data elements of the historic data may be used to compute the at least one threshold value. In the latter case, the at least one threshold value may reflect an average or mean energy consumption or reduction in capacity of the energy storage per usage session.

In an exemplary implementation, the control circuitry may be configured to compute or calculate the threshold value based on a current value of a running average of the energy consumption per usage session and/or duration of a usage session, for example based on processing the corresponding data elements of the historic data. Accordingly, the at least one threshold value may be computed by the control circuitry based on determining a current value of a running average of the energy consumption per usage session and/or duration of a usage session. Accordingly, the current value of the running average of the energy consumption per usage session may correspond to the mean or average amount of energy consumed in the one or more usage sessions considered in the running average. Alternatively or additionally, the current value of the running average of the duration of the usage session may correspond to the mean or average duration of the usage sessions considered in the running average.

Generally, computing the threshold value based on computing a value of a running average may allow to reduce a data volume stored at the aerosol-generating device. For example, only the current value of the running average may be stored in a data storage of the aerosol-generating device, without necessarily storing the historic data. In particular, the current value of the running average may even be stored in Random Access Memory (RAM) only.

For instance, the control circuitry may be configured to calculate the current value of the running average of the energy consumed or energy consumption based on the preceding or last value of the running average of the energy consumption, which may optionally be stored in and/or retrieved from the data storage or RAM of the aerosol-generating device, and based on the amount of energy used or consumed in the last or previous usage session.

Optionally, the last value of the running average may be multiplied by a certain factor or constant, C, and added to the amount of energy used or consumed in the last usage session. Further, the result of this computation may be divided or normalized by the constant or factor, C, plus one to provide a new or the current value of the running average. The constant or factor, C, can, for example range from about 2 to several tens, for example the constant or factor C may be about 9.

Mathematically expressed, the current value of the running average, CV, may be computed as CV=(LV*C+duration of current usage session))/(C+1), wherein C is the constant or factor, and LV is the last or preceding value of the running average. Initially (e.g. when the device is used for the first time), where there is no last or preceding value per se a dummy value may be used. The current value of the running average for the energy consumption per usage session may then be used as the at least one threshold value, or may be used to compute the at least one threshold value.

Alternatively or additionally, the control circuitry may be configured to calculate the current value of the running average of the duration of a usage session based on the preceding or last value of the running average of the duration of a usage session, which may optionally be stored in and/or retrieved from the data storage or RAM of the aerosol-generating device, and based on the duration of the last or previous usage session.

Optionally, the last value of the running average may be multiplied by a certain factor or constant, C, and added to the duration of the last usage session. Further, the result of this computation may be divided or normalized by the constant or factor, C, plus one to provide a new or the current value of the running average. The constant or factor, C, can, for example range from about 2 to several tens, for example the constant or factor C may be about 9.

Mathematically expressed, the current value of the running average, CV, may be computed as CV=(LV*C+duration of current usage session))/(C+1), wherein C is the constant or factor, and LV is the last, previous or preceding value of the running average. The current value of the running average of the duration of a usage session may then be used as the at least one threshold value, or may be used to compute the at least one threshold value.

In a further optional implementation, the control circuitry may be configured to compute the at least one threshold value based on computing a plurality of values of a plurality of running averages of the amount of energy consumed during a usage session, energy consumption per usage session, and/or of the duration of a usage session. Therein, different running averages of the plurality of running averages may differ in terms of the number of usage sessions and/or time period considered for the particular running average, respectively for computing the corresponding values.

For example, a first value of a first running average for the energy consumed per usage session and/or duration of a usage session may be computed based on a first number of previous usage sessions, and a second value of a second running average for the energy consumed per usage session and/or duration of a usage session may be computed based on a second number of previous usage sessions. Therein, the first number of previous usage sessions may differ from the second number of previous usage sessions. In particular, the first number of previous usage sessions may be smaller than the second number of previous usage sessions.

In a non-limiting example, the first number of previous usage sessions may be about 2 to about 20 previous or past usage sessions, and the second number of previous usage sessions may be about 200 to about 20000 usage sessions. Accordingly, the first running average may consider only the last few usage sessions, such that the corresponding value reflects the short-term behaviour of the user. The second running average, on the other hand, may reflect the user's long-term behaviour or habit.

In an example, the control circuitry may be configured to compute the at least one threshold value based on computing an average energy per usage session and/or an average duration of a usage session based on multiplying the second value of the second running average, Value_2, with a constant or variable factor, C, and based on multiplying the first value of the first running average, Value_1, with one minus the constant or variable factor, C.

Mathematically expressed, the threshold value T may be computed as T=C+Value_2+(1−C)*Value_1, wherein C is the constant or variable factor, Value_1 is the first value of the first running average and Value_2 is the second value of the second running average. The constant or variable factor C may for example be between 0.2 and 0.9, such as 0.7.

Depending on whether the first and second values correspond to average energy consumption or duration, also the threshold value may be given in terms of a mean or average threshold energy consumption or duration. Other parameters, such as relative energy reduction per usage session may be used instead or in addition.

Optionally, the number of usage sessions considered by the control circuitry for computation of the at least one threshold value may be user-definable, for example based on providing one or more user inputs.

The control circuitry may be configured to determine, based on historic data indicative of an operation of the aerosol-generating device in a pause mode, an amount of energy used during a single usage pause, and to adjust the at least one threshold value based on the determined amount of energy. For example, one or more of start time, stop time, duration, energy consumption, reduction in amount of energy stored in the energy storage, reduction in capacity of the energy storage, temperature, temperature profile, an indication of an interruption of a usage session by a pause mode, a number of pause modes or usage pauses in a usage session may be stored or recorded by the control circuitry for one or more past or previous usage pauses or pause modes initiated by the user. The historic data may for example be stored at a data storage of the aerosol-generating device.

The control circuitry may further be configured to process and/or analyse one or more of the data elements of the historic data and determine one or more threshold values for evaluation with the storage status of the energy storage. For instance, one or more threshold values related to an energy consumption per usage pause (or pause mode) and/or a change in capacity per usage pause (or pause mode) may be determined by the control circuitry. Hence, the consumption behaviour of the user, as reflected by the historic data, may be used to dynamically adjust the at least one threshold value and/or to adapt a number of usage pauses allowed or granted to the user per fully charged energy storage.

In an example, the control circuitry may be configured to determine or compute the at least one threshold value based on predefined number of previous or past usage pauses or pause modes. For example, a number of data elements of the historic data corresponding to the predefined number of previous or past usage pauses or pause modes may be processed and/or analysed by the control circuitry to derive or compute the at least one threshold value for evaluation with the storage status of the energy storage. In a non-limiting example, only the last usage pause or corresponding data element of the historic data may be used to compute the at least one threshold value. Alternatively, two or more, for example between two and 20, preferably between two and 16 previous or past usage pauses or corresponding data elements of the historic data may be used to compute the at least one threshold value. In the latter case, the at least one threshold value may reflect an average or mean energy consumption or reduction in capacity of the energy storage per usage pause. Optionally, the number of usage pauses considered by the control circuitry for computation of the at least one threshold value may be user-definable, for example based on providing one or more user inputs.

Optionally, the control circuitry may be configured to derive or compute, based on the historic data, a first threshold value indicative of an energy consumption during one or more usage sessions and a second threshold value indicative of an energy consumption during one or more usage pauses. Alternatively or additionally, first and second threshold values for the duration of a usage session or other parameters may be computed.

In an example, the at least one threshold value may be indicative of a threshold energy required for operating the aerosol-generating device during at least one usage pause interrupting a usage session. Non-limiting and exemplary threshold values may range from about 2 mAh to about 80 mAh, for example about 10 mAh to about 65 mAh. Other values may be used, though.

In an exemplary implementation, the at least one threshold value may be received from a computing device or companion device communicatively couplable to the aerosol-generating device. The companion device may also be referred to herein as receiving device.

For example, the aerosol-generating device may be couplable, for example operatively and/or communicatively couplable, to a companion or receiving device for supplying electrical energy to the energy storage, wherein the at least one threshold value may be received from the companion or receiving device upon coupling the aerosol-generating device to the companion device.

The companion or receiving device may be configured to at least partially receive the aerosol-generating device, for example for charging the aerosol-generating device and/or or for storing the aerosol-generating device. The aerosol-generating device may be configured for wireless charging by the companion device. For example, the companion device and the aerosol-generating device may be inductively coupled to charge the energy storage of the aerosol-generating device. Alternatively or additionally, the control circuitry may be configured to establish an electrical connection with the companion device to charge the energy storage. Accordingly, the aerosol-generating device may be configured for being operatively coupled to the companion for charging the energy storage of the aerosol-generating device.

Alternatively or additionally, the companion device may comprise a communication interface for communicatively coupling the companion device to the aerosol-generating device, and for example for transmitting the at least one threshold value or a corresponding control signal from the companion device to the aerosol-generating device. Optionally, communicative coupling of the aerosol-generating device and the companion device may be established upon operative coupling of the aerosol-generating device with the companion device to charge the aerosol-generating device, and vice versa.

In an example, the companion device may comprise a user interface for receiving one or more user inputs, for example to define the at least one threshold value or information allowing to derive the at least one threshold value, such as a number usage sessions and/or usage pauses per fully charged energy storage, a duration of a usage session and/or usage pause, at the companion device, which may be transmitted to the aerosol-generating device.

Alternatively or additionally, the companion device may be communicatively coupled to a computing device, such as a server, server network, computer, tablet, smart phone or a mobile device, and the at least one threshold value or a corresponding control signal may be received by the companion device from the computing device and then transmitted to the aerosol-generating device. Alternatively or additionally, historic data may be transmitted and/or exchanged between the aerosol-generating device and the companion device, between the computing device and the aerosol-generating device, and/or between the computing device and the companion device.

In an exemplary implementation, the at least one threshold value may be indicative of a time period or duration of a single usage session. Alternatively or additionally, the at least one threshold value may be indicative of a time period or duration of a single usage pause. Accordingly, the at least one threshold value may be time-based. Optionally, also the storage status of the energy storage may be time-based, for example it may be indicative of a time the device may be operated with a single fully charged energy storage, after the energy storage has been charged the last time, per complete charge of the energy storage and/or between two consecutive charging events to generate aerosol in one or more usage sessions and/or in one or more usage pauses. Based on evaluating the time-based storage status against the threshold value during operation, the at least one device function, for example a particular number of usage sessions and/or usage pauses as reflected by the threshold value, may be granted or allowed to the user with a single fully charged energy store and/or before enforcing re-charging of the energy storage. Alternatively or additionally, the time-based threshold value may be converted to an energy or capacity based value or other quantity, and compared to the storage status or a quantity derived therefrom.

In another example, the at least one threshold value may be indicative of an average energy consumption per usage session. Alternatively or additionally, the at least one threshold value may be indicative of an average reduction in capacity of the energy storage per usage session.

Alternatively or additionally, the at least one threshold value may be indicative of an average energy consumption per usage pause interrupting one or more usage sessions. Alternatively or additionally, the at least one threshold value may be indicative of an average reduction in capacity of the energy storage per usage pause or pause mode.

As noted above, the control circuitry may, in an example, be configured to compute the at least one threshold value based on historic data and to store the computed at least one threshold value in a data storage of the aerosol-generating device. Alternatively or additionally, the control circuitry may be configured to retrieve historic data from a data storage of the aerosol-generating device and/or from an external data source, the historic data being indicative of one or more of a number of usage sessions the aerosol-generating device has been operated to generate aerosol, a number of usage pauses the aerosol-generating device has been operated in a pause mode interrupting one or more usage sessions, a duration of one or more usage sessions, a duration of one or more usage pauses interrupting one or more usage sessions, energy consumption during one or more usage sessions, energy consumption during one or more usage pauses, total energy consumption, charging data related to charging of the energy storage in one or more charging cycles, remaining energy in the energy storage before re-charging in one or more charging cycles, and energy consumption per usage session.

In yet another exemplary implementation, the control circuitry may be configured to determine a health status of the energy storage based on historic data indicative of an operation of the aerosol-generating device to generate aerosol. Accordingly, the control circuitry may be configured to determine the storage status based on processing the historic data. For instance, the control circuitry may determine or compute the health status of the energy storage based one or more of a number of usage sessions and/or usage pauses the device has been operated, total energy consumption, operation time or time of operation, current maximum capacity, relative reduction in capacity with respect to an initial maximum capacity and/or other information stored in the historic data, as also described in more detail hereinabove and hereinbelow.

In an example, the control circuitry may be configured to compute the at least one threshold value based on data received from a computing device or a companion device communicatively couplable to the aerosol-generating device. For example the historic data or data related to one or more user inputs, may be received from the computing device and/or companion or receiving device.

The control circuitry may be configured to compute the at least one threshold value based on historic data related to one or more previous usage sessions and/or one or more previous usage pauses. Optionally, the control circuitry may be configured to compute an average or mean energy consumption of energy consumed during a plurality of usage sessions and/or usage pauses based on historic data indicative of an operation of the aerosol-generating device during one or more usage sessions and/or one or more usage pauses. Alternatively or additionally, a mean or average duration of a usage session or one or more other parameters may be computed.

In an exemplary configuration, the aerosol-generating device may comprise at least a part of a heating element, heating arrangement or a heating circuit coupled to the energy storage and configured to heat at least a portion of an aerosol-generating article. Accordingly, at least a part of the heating element, heating arrangement and/or at least a part of a heating circuit may be integrated in the aerosol-generating device.

For example, a resistive blade, a resonator, an exciter coil, a microwave generator or other type of heating element may be at least partially arranged and/or integrated in the aerosol-generating device.

Alternatively or additionally, the aerosol-generating device may be couplable to an aerosol-generating article, which includes at least a part of a heating element, heating arrangement or heating circuit, wherein the energy storage may be configured to supply electrical energy to the at least one heating element of the aerosol-generating article to generate aerosol.

For instance, a resistive blade, a resonator, an exciter coil, a microwave generator or other type of heating element may be at least partially arranged and/or integrated in the aerosol-generating article and powered or supplied with electrical energy drawn from the energy storage of the aerosol-generating device.

According to a further aspect of the disclosure, there is provided an aerosol-generating system comprising an aerosol-generating device, as described hereinabove and hereinbelow, and an aerosol-generating article couplable or coupled to the aerosol-generating device to generate aerosol. The aerosol-generating device may generate aerosol, for example, by supplying electrical energy to one or more aerosol generators for generating aerosol from the aerosol-generating article. Any disclosure presented hereinabove and hereinbelow with reference to the aerosol-generating device according to one or more aspects of the present disclosure and/or the aerosol-generating article equally applies to the aerosol-generating system, and vice versa.

The energy storage and/or control circuitry may be configured to supply electrical energy to at least one aerosol-generating means or aerosol generator for generating aerosol from at least a portion of an aerosol-generating article couplable to the aerosol-generating device. Exemplary aerosol generators or means may include one or more heating elements, one or more heat sources, one or more vibrating elements, one or more vibrating meshes, and one or more spraying devices.

For example, the energy storage and/or control circuitry may be configured to supply electrical energy to at least one heating element to generate aerosol from an aerosol-generating article couplable to the aerosol-generating device, for example based on heating at least a portion of the aerosol-generating article.

A further aspect of the disclosure relates to use of an aerosol-generating device and/or system, as described hereinabove and hereinbelow.

Yet another aspect of the disclosure relates to a method of operating an aerosol-generating device. The aerosol-generating device operated in accordance with the method may be an aerosol-generating device, as described hereinabove and hereinbelow with reference to one or more aspects of the present disclosure. Accordingly, any feature function and/or element described hereinabove and hereinbelow with reference to the aerosol-generating device may be a feature, function, element and/or step of the method of operating the aerosol-generating device, and vice versa. Alternatively or additionally, the method may relate to a method of operating an aerosol-generating system, as described hereinabove and hereinbelow.

The aerosol-generating device operated in accordance with the method of the present disclosure includes an energy storage configured to supply electrical energy to control circuitry for generating aerosol from an aerosol-generating article. The control circuitry may be operatively coupled to the energy storage. The energy storage may supply electrical energy to a heating element to heat at least a portion of an aerosol-generating article couplable to the aerosol-generating device. The method comprises at least:
determining, with the control circuitry, a storage status of the energy storage indicative of an amount of electrical energy currently stored and/or storable in the energy storage;
evaluating the determined storage status with respect to at least one threshold value associated with heating of the aerosol-generating article; and
enabling or disabling, based on the evaluation, at least one device function associated with heating of the aerosol-generating article.

A further aspect of the present disclosure relates to a method of operating an aerosol-generating device, the method comprising at least:
determining, with the control circuitry, a storage status of the energy storage indicative of at least one of an amount of electrical energy currently stored and an amount of electrical energy currently storable in the energy storage;
evaluating the determined storage status with respect to at least one threshold value, wherein the at least one threshold value correlates with a threshold energy required for performing a main heating function of the aerosol-generating device for heating the aerosol-generating article at or above a predetermined heating temperature to generate aerosol in at least one usage session, and for performing at least one auxiliary device function of the aerosol-generating device different than the main heating function; and
enabling or disabling, based on the evaluation, at least one of the main heating function and the at least one auxiliary device function

A further aspect of the present disclosure relates to a method of operating an aerosol-generating device, the method comprising at least:
determining, with the control circuitry, a storage status including a health status of the energy storage indicative of an amount of electrical energy currently storable in the energy storage;
evaluating the determined storage status with respect to at least one threshold value associated with at least one device function of the aerosol-generating device; and
enabling or disabling, based on the evaluation, at least one device function.

A further aspect of the present disclosure relates to a method of operating an aerosol-generating device, the method comprising at least:
determining, with the control circuitry, a storage status of the energy storage indicative of at least one of an amount of electrical energy currently stored and an amount of electrical energy currently storable in the energy storage;
evaluating the determined storage status with respect to at least one threshold value associated with at least one device function of the aerosol-generating device, wherein the at least threshold value is adjustable; and
enabling or disabling, based on the evaluation, at least one device function.

Any one of the methods described with reference to one or an aspect of the present disclosure can be combined with one or more steps of one or more further methods according to one or more further aspects of the present disclosure.

The energy storage and/or control circuitry may be configured to supply electrical energy to at least one aerosol-generating means or aerosol generator for generating aerosol from at least a portion of an aerosol-generating article couplable to the aerosol-generating device. Exemplary aerosol generators or means may include one or more heating elements, one or more heat sources, one or more vibrating elements, one or more vibrating meshes, and one or more spraying devices.

For example, the energy storage and/or control circuitry may be configured to supply electrical energy to at least one heating element to generate aerosol from an aerosol-generating article couplable to the aerosol-generating device, for example based on heating at least a portion of the aerosol-generating article.

In an example, determining the storage status may include determining at least one of a current energy level of electrical energy stored in the energy storage, and a health status of the energy storage indicative of the amount of electrical energy currently storable in the energy storage.

In yet another example, evaluating the determined storage status may include comparing or intercomparing the determined storage status to the at least one threshold value. Optionally, the at least one device function may be enabled by the control circuitry upon determining, based on the evaluation, that a current energy level of electrical energy stored and/or storable in the energy storage is sufficient for operating the aerosol-generating device during at least one usage session to generate inhalable aerosol based on heating the aerosol-generating article to a temperature at or above a predetermined heating temperature.

In an example, the method may further comprise enabling a main heating function upon determining, based on the evaluation, that a current energy level of electrical energy stored and/or storable in the energy storage is sufficient for performing the main heating function during at least one usage session to generate inhalable aerosol. Alternatively or additionally, the method may further comprise enabling the at least one device function upon determining, based on the evaluation, that a current energy level of electrical energy stored and/or storable in the energy storage is sufficient for completing a current usage session and/or for completing a predefined number of usage sessions to generate inhalable aerosol based on heating the aerosol-generating article.

In a further example, the method may comprise enabling the at least one device function upon determining, based on the evaluation, that a current energy level of electrical energy stored and/or storable in the energy storage is sufficient for operating the aerosol-generating device during at least one usage pause in a pause mode interrupting a usage session.

Alternatively or additionally the method may further comprise enabling at least one auxiliary device function upon determining, based on the evaluation, that a current energy level of electrical energy stored and/or storable in the energy storage is sufficient for performing the at least one auxiliary device function of the aerosol-generating device.

The method may further comprise enabling at least one of a main heating function of the aerosol-generating device to generate aerosol in at least one usage session based on heating the aerosol-generating article to or above the predetermined heating temperature, and at least one auxiliary device function of the aerosol-generating device different than, other than and/or non-related to the main heating function or to heating the aerosol-generating device during a usage session to generate aerosol.

The method further comprise enabling one or more auxiliary device functions including one or more of operating the aerosol-generating device at reduced energy consumption with respect to operation during a usage session to generate aerosol, heating the at least one heating element and/or the aerosol-generating article to a temperature below a heating temperature sufficient to generate aerosol, heating the at least one heating element and/or the aerosol-generating article to a temperature above room temperature and below a heating temperature sufficient to generate aerosol, heating the at least one heating element and/or the aerosol-generating article to a temperature above room temperature and below a heating temperature sufficient to generate aerosol for a predefined period of time, operating a user interface of the aerosol-generating device, operating a communication circuitry of the aerosol-generating device for communicatively coupling the aerosol-generating device to a computing device or receiving device, operating the device in a usage pause or pause mode, operating a sensor of the aerosol-generating device, activating or deactivating haptic control, and operating a biometric sensor of the aerosol-generating device for user authentication.

The method may further comprise enabling a) a main heating function of the aerosol-generating device in at least one usage session to generate aerosol, and b) at least one auxiliary device function of the aerosol-generating device different than, other than, and/or non-related to the main heating function of the aerosol-generating device, upon determining, based on the evaluation, that a current energy level of electrical energy stored and/or storable in the energy storage is sufficient for performing the main heating function and the at least one auxiliary device function of the aerosol-generating device.

Alternatively or additionally, upon determining, based on the evaluation, that a current energy level of electrical energy stored and/or storable in the energy storage is insufficient for performing the main heating function and the at least one auxiliary device function of the aerosol-generating device, the main heating function and/or the auxiliary device function may be disabled or prevented from being initiated by the user.

Optionally, the method may further comprise triggering re-charging of the energy storage based on the evaluation of the determined storage status with respect to the at least one threshold value, for example upon determining that a current energy level of electrical energy stored and/or storable in the energy storage is insufficient for performing the main heating function and the at least one auxiliary device function of the aerosol-generating device.

In an example, determining the storage status may include determining a current energy level of electrical energy stored and/or storable in the energy storage relative to a current maximum capacity of the energy. Optionally, an initial maximum capacity of the energy storage may be determined by the control circuitry.

In an example, the method may further comprise determining whether the energy storage has been charged to a predefined charging stage in a previous charging event.

In yet another example, determining the storage status may include determining an age or health status of the energy storage.

The method may further comprise determining one or more of a current maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated to generate aerosol, charging data related to charging of the energy storage in one or more charging cycles, and a total energy consumption of the aerosol-generating device.

Optionally, determining the storage status may include determining a current maximum capacity of the energy storage indicative of a maximum amount of energy storable in the energy storage and determining a current energy level or amount of electrical energy currently stored in the energy storage.

As an example, the at least one threshold value may include a first threshold value for a current energy level or amount of energy available in the energy storage, and a second threshold value for a current maximum capacity of the energy storage indicative of a maximum amount of energy currently storable in the energy storage.

Optionally, the method may further comprise one or more of comparing a determined current energy level to the first threshold value, and comparing a determined current maximum capacity of the energy storage to the second threshold value.

In yet another example, the method may further comprise adjusting, modifying, changing, replacing and/or altering the at least one threshold value.

For instance, the at least one threshold value may be adjusted based on historic data indicative of a past usage of the aerosol-generating device and/or indicative of a consumption behaviour of a user.

Alternatively or additionally, the method may further comprise computing the at least one threshold value, based on historic data indicative of an operation of the aerosol-generating device to generate aerosol, an amount of energy used in a single usage session, and adjusting the at least one threshold value based on the determined amount of energy used in the single usage session.

In an example, the method may further comprise determining, based on historic data indicative of an operation of the aerosol-generating device to generate aerosol, a maximum number of usage sessions the aerosol-generating device is usable with a fully charged energy storage, and adjusting the at least one threshold value based on the determined maximum number of usage sessions.

The method may further comprise indicating the determined maximum number of usage sessions to the user based on controlling a user interface of the aerosol-generating device. For instance a number of LEDs corresponding to the number of usage sessions or experiences available to the user in the current status of the energy storage may be lit or controlled by the control circuitry.

The method may further comprise enabling or allowing use of the aerosol-generating device for a subsequent usage session independent of a number of previous usage sessions, the aerosol-generating device has been used since a previous charging process.

In an example, the at least one threshold value may be adjusted and/or varied based on a user input.

Alternatively or additionally, the method may further comprise computing the at least one threshold value based on historic data collected in one or more past usage sessions.

Alternatively or additionally, the method may further comprise determining, based on historic data indicative of an operation of the aerosol-generating device in a pause mode, an amount of energy used during a single usage pause, and adjusting the at least one threshold value based on the determined amount of energy.

The method may further comprise determining a health status, an age and/or the storage status of the energy storage based on historic data indicative of an operation of the aerosol-generating device to generate aerosol during one or more usage sessions and/or during one or more usage pauses or pause modes.

Optionally, the method may further comprise computing the at least one threshold value based on data received from a computing device or a receiving device communicatively couplable to the aerosol-generating device.

For instance, the at least one threshold value may be computed based on historic data related to one or more previous usage sessions. Alternatively or additionally, an average energy consumption of energy consumed during a plurality of usage sessions may be computed based on historic data indicative of an operation of the aerosol-generating device to generate aerosol, and optionally used as threshold value.

A further aspect of the disclosure relates to a computer program, which when executed by an aerosol-generating device or an aerosol-generating system, instructs the aerosol-generating device or system to perform steps of one or more methods according to one or more aspects of the present disclosure, as described hereinabove and hereinbelow.

A further aspect of the disclosure relates to a computer-readable medium, for example a non-transitory computer-readable medium, storing a computer program, which when executed by an aerosol-generating device or an aerosol-generating system, instructs the aerosol-generating device or system to perform steps of one or more methods according to one or more aspects of the present disclosure, as described hereinabove and hereinbelow.

It is emphasized that any feature, step, function, element, technical effect and/or advantage described herein with reference to one aspect of the disclosure equally applies to any other aspect of the disclosure.

Below, there is provided a non-exhaustive list of non-limiting examples. Any one or more of the features of these examples may be combined with any one or more features of another example, embodiment, or aspect described herein.

Example 1a: An aerosol-generating device, comprising:
control circuitry and an energy storage configured to supply electrical energy to the control circuitry for generating aerosol from an aerosol generating article;
wherein the control circuitry is configured to:
determine a storage status of the energy storage indicative of an amount of electrical energy currently stored and/or storable in the energy storage;
   evaluate the determined storage status with respect to at least one threshold value associated with at least one device function of the aerosol-generating device, for example associated with heating of the aerosol-generating article; and
enable or disable, based on the evaluation, at least one device function.

Example 1b: An aerosol-generating device, comprising:
control circuitry and an energy storage configured to supply electrical energy to the control circuitry for generating aerosol from an aerosol generating article;
wherein the control circuitry is configured to:
   determine a storage status of the energy storage indicative of at least one of an amount of electrical energy currently stored and an amount of electrical energy currently storable in the energy storage;
   evaluate the determined storage status with respect to at least one threshold value, wherein the at least one threshold value correlates with a threshold energy required for performing a main heating function of the aerosol-generating device for heating the aerosol-generating article at or above a predetermined heating temperature to generate aerosol in at least one usage session, and for performing at least one auxiliary device function of the aerosol-generating device different than the main heating function; and
   enable or disable, based on the evaluation, at least one of the main heating function and the at least one auxiliary device function.

Example 1c: An aerosol-generating device, comprising:
control circuitry and an energy storage configured to supply electrical energy to the control circuitry for generating aerosol from an aerosol-generating article;
wherein the control circuitry is configured to:
   determine a storage status including a health status of the energy storage indicative of an amount of electrical energy currently storable in the energy storage;
   evaluate the determined storage status with respect to at least one threshold value associated with at least one device function of the aerosol-generating device; and
   enable or disable, based on the evaluation, at least one device function.

Example 1d: An aerosol-generating device, comprising:
control circuitry and an energy storage configured to supply electrical energy to the control circuitry for generating aerosol from an aerosol-generating device;
wherein the control circuitry is configured to:
   determine a storage status of the energy storage indicative of at least one of an amount of electrical energy currently stored and an amount of electrical energy currently storable in the energy storage;
   evaluate the determined storage status with respect to at least one threshold value associated with at least one device function of the aerosol-generating device, wherein the at least threshold value is adjustable; and
   enable or disable, based on the evaluation, at least one device function.

Example 1e: The aerosol-generating device according to any one of the preceding examples, wherein the energy storage is configured to supply electrical energy to one or more aerosol generators for generating aerosol from at least a portion of an aerosol-generating article couplable to the aerosol-generating device.

Example 1f: The aerosol-generating device according to any one of the preceding examples, wherein the energy storage is configured to supply electrical energy to at least one heating element to heat at least a portion of the aerosol-generating article couplable to the aerosol-generating device.

Example 1g: The aerosol-generating device according to any one of the preceding examples, wherein the control circuitry is operatively coupled to the energy storage.

Example 2: The aerosol-generating device according to any one of the preceding examples, wherein the determined storage status includes at least one of a current energy level of electrical energy stored in the energy storage, and a health status of the energy storage indicative of an amount of electrical energy currently storable in the energy storage.

Example 3: The aerosol-generating device according to any one of the preceding examples, wherein evaluating the determined storage status includes comparing the determined storage status to the at least one threshold value, preferably wherein the determined storage status is expressed as a value.

Example 4: The aerosol-generating device according to any one of the preceding examples, wherein the at least one threshold value is indicative of a threshold energy required for operating the aerosol-generating device during at least one usage session to generate inhalable aerosol based on heating the aerosol-generating article to a temperature at or above a predetermined heating temperature to generate aerosol.

Example 5: The aerosol-generating device according to any one of the preceding examples, wherein the control circuitry is configured to enable the at least one device function upon determining, based on the evaluation, that a current energy level of electrical energy stored and/or storable in the energy storage is sufficient for operating the aerosol-generating device during at least one usage session to generate inhalable aerosol based on heating the aerosol-generating article to a temperature at or above a predetermined heating temperature to generate aerosol.

Example 6: The aerosol-generating device according to any one of the preceding examples, wherein the at least one threshold value is indicative of a threshold energy required for performing a main heating function of the aerosol-generating device during at least one usage session, the main heating function involving supplying electrical energy to the at least one heating element to heat at least a part of the aerosol-generating article to a temperature at or above a predetermined heating temperature to generate aerosol.

Example 7: The aerosol-generating device according to any one of the preceding examples, wherein the control circuitry is configured to enable a main heating function upon determining, based on the evaluation, that a current energy level of electrical energy stored and/or storable in the energy storage is sufficient for performing the main heating function during at least one usage session to generate inhalable aerosol.

Example 8: The aerosol-generating device according to any one of the preceding examples, wherein the at least one threshold value is indicative of a threshold energy required for completing a current usage session to generate inhalable aerosol based on heating the aerosol-generating article to a temperature at or above a predetermined heating temperature to generate aerosol; and/or wherein the at least one threshold value is indicative of a threshold energy required for completing a predefined number of usage sessions to generate inhalable aerosol based on heating the aerosol-generating article to a temperature at or above a predetermined heating temperature to generate aerosol.

Example 9: The aerosol-generating device according to any one of the preceding examples, wherein the control circuitry is configured to enable the at least one device function upon determining, based on the evaluation, that a current energy level of electrical energy stored and/or storable in the energy storage is sufficient for completing a current usage session and/or for completing a predefined number of usage sessions to generate inhalable aerosol based on heating the aerosol-generating article to a temperature at or above a predetermined heating temperature to generate aerosol.

Example 10: The aerosol-generating device according to any one of the preceding examples, wherein the at least one threshold value is indicative of a threshold energy required for operating the aerosol-generating device during at least one usage pause in a pause mode interrupting a usage session.

Example 11: The aerosol-generating device according to any one of the preceding examples, wherein the control circuitry is configured to enable the at least one device function upon determining, based on the evaluation, that a current energy level of electrical energy stored and/or storable in the energy storage is sufficient for operating the aerosol-generating device during at least one usage pause in a pause mode interrupting a usage session.

Example 12: The aerosol-generating device according to any one of the preceding examples, wherein the at least one threshold value is indicative of a threshold energy required for performing at least one auxiliary device function of the aerosol-generating device.

Example 13 The aerosol-generating device according to any one of the preceding examples, wherein the control circuitry is configured to enable at least one auxiliary device function upon determining, based on the evaluation, that a current energy level of electrical energy stored and/or storable in the energy storage is sufficient for performing the at least one auxiliary device function of the aerosol-generating device.

Example 14: The aerosol-generating device according to example 13, wherein the auxiliary device function is non-related to or differs from heating the aerosol-generating device during a usage session to generate aerosol based on heating the aerosol-generating article to a temperature at or above a predetermined heating temperature to generate aerosol; and/or
wherein the auxiliary device function is associated with a pause mode interrupting a usage session.

Example 15: The aerosol-generating device according to any one of the preceding examples, wherein the at least one device function includes at least one of a main heating function of the aerosol-generating device to generate aerosol in at least one usage session based on heating the aerosol-generating article to a temperature at or above a predetermined heating temperature to generate aerosol, and at least one auxiliary device function of the aerosol-generating device, which differs from the main heating function.

Example 16: The aerosol-generating device according to any one of examples 12 to 15, wherein the auxiliary device function includes one or more of:
operating the aerosol-generating device at reduced energy consumption with respect to operation during a usage session to generate aerosol;
heating the at least one heating element and/or the aerosol-generating article to a temperature below a heating temperature sufficient to generate aerosol;
heating the at least one heating element and/or the aerosol-generating article to a temperature above room temperature and below a heating temperature sufficient to generate aerosol;
heating the at least one heating element and/or the aerosol-generating article to a temperature above room temperature and below a heating temperature sufficient to generate aerosol for a predefined period of time;
activating or deactivating haptic control of the aerosol-generating device;
operating a user interface of the aerosol-generating device;
operating a communication circuitry of the aerosol-generating device for communicatively coupling the aerosol-generating device to a computing device or receiving device;
operating a sensor of the aerosol-generating device; and
operating a biometric sensor of the aerosol-generating device for user authentication.

Example 17: The aerosol-generating device according to any one of the preceding examples, wherein the at least one threshold value correlates with a threshold energy required for a) performing a main heating function of the aerosol-generating device in at least one usage session to generate aerosol, and b) for performing an auxiliary device function of the aerosol-generating device different than the main heating function of the aerosol-generating device.

Example 18: The aerosol-generating device according to any one of the preceding examples, wherein the at least one threshold value correlates with a threshold energy required for operating the aerosol-generating device a) during at least one usage session in an aerosol-releasing mode generating inhalable aerosol based on heating the aerosol-generating article to a temperature at or above a predetermined heating temperature, and b) during a usage pause in a pause mode interrupting the at least one usage session.

Example 19: The aerosol-generating device according to any one of the preceding examples, wherein the control circuitry is configured to enable a) a main heating function of the aerosol-generating device in at least one usage session to generate aerosol, and b) at least one auxiliary device function of the aerosol-generating device different than the main heating function of the aerosol-generating device, upon determining, based on the evaluation, that a current energy level of electrical energy stored and/or storable in the energy storage is sufficient for performing the main heating function and the at least one auxiliary device function of the aerosol-generating device.

Example 20: The aerosol-generating device according to any one of the preceding examples, wherein the control circuitry is configured to trigger or request re-charging of the energy storage based on the evaluation of the determined storage status with respect to the at least one threshold value.

Example 21: The aerosol-generating device according to any one of the preceding examples, wherein the at least one threshold value is indicative of a fraction of a current maximum capacity of the energy storage, in particular about 70% to about 100% of the current maximum capacity, preferably at least about 90% of the current maximum capacity.

Example 22: The aerosol-generating device according to any one of the preceding examples, wherein determining the storage status includes determining a current energy level of electrical energy stored and/or storable in the energy storage relative to a current maximum capacity of the energy.

Example 23: The aerosol-generating device according to any one of the preceding examples, wherein the control circuitry is configured to determine a current maximum capacity of the energy storage.

Example 24: The aerosol-generating device according to any one of the preceding examples, wherein the at least one threshold value is indicative of a fraction of an initial maximum capacity of the energy storage, in particular between about 35% and about 75% of the initial maximum capacity.

Example 25: The aerosol-generating device according to any one of the preceding examples, wherein the control circuitry is configured to determine an initial maximum capacity of the energy storage.

Example 26: The aerosol-generating device according to any one of the preceding examples, wherein the at least one threshold value is indicative of a predefined charging stage the energy storage has reached in a previous charging event, in particular a constant voltage stage of about 3.65 V charging voltage.

Example 27: The aerosol-generating device according to any one of the preceding examples, wherein the control circuitry is configured to determine whether the energy storage has been charged to a predefined charging stage in a previous charging event.

Example 28: The aerosol-generating device according to any one of the preceding examples, wherein determining the storage status includes determining a health status of the energy storage; and/or
wherein the control circuitry is configured to determine a health status of the energy storage.

Example 29: The aerosol-generating device according to any one of the preceding examples, wherein the at least one threshold value is indicative of a threshold health status and/or threshold age of the energy storage.

Example 30: The aerosol-generating device according to any one of the preceding examples, wherein determining the storage status includes determining an age of the energy storage

Example 31: The aerosol-generating device according to any one of the preceding examples, wherein the control circuitry is configured to determine a health status of the energy storage based on determining one or more of a current maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated to generate aerosol, charging data related to charging of the energy storage in one or more charging cycles, and a total energy consumption of the aerosol-generating device.

Example 32: The aerosol-generating device according to any one of the preceding examples, wherein the control circuitry is configured to determine one or more of a current maximum capacity of the energy storage, an initial maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated to generate aerosol, charging data related to charging of the energy storage in one or more charging cycles, and a total energy consumption of the aerosol-generating device.

Example 33: The aerosol-generating device according to any one of the preceding examples, wherein determining the storage status includes determining a current maximum capacity of the energy storage indicative of a maximum amount of energy storable in the energy storage and determining a current energy level or amount of electrical energy currently stored in the energy storage.

Example 34: The aerosol-generating device according to any one of the preceding examples, wherein the at least one threshold value includes a first threshold value for a current energy level or amount of energy available in the energy storage, and a second threshold value for a current maximum capacity of the energy storage indicative of a maximum amount of energy currently storable in the energy storage; and wherein the control circuitry is configured to compare a determined current energy level to the first threshold value and to compare a determined current maximum capacity of the energy storage to the second threshold value.

Example 35: The aerosol-generating device according to any one of the preceding examples, wherein the at least one threshold value is adjustable; and/or wherein the control circuitry is configured to adjust the at least one threshold value.

Example 36: The aerosol-generating device according to any one of the preceding examples, wherein the control circuitry is configured to adjust the at least one threshold value based on historic data indicative of a past usage of the aerosol-generating device and/or indicative of a consumption behaviour of a user.

Example 37: The aerosol-generating device according to any one of the preceding examples, wherein the control circuitry is configured to compute, based on historic data indicative of an operation of the aerosol-generating device to generate aerosol, an amount of energy used in one or more usage sessions, for example in a single usage session, and to adjust the at least one threshold value based on the determined amount of energy used in the one or more usage sessions, for example in the single usage session.

Example 38: The aerosol-generating device according to any one of the preceding examples, wherein the at least one threshold value is indicative of a threshold energy required for operating the aerosol-generating device during at least one usage session to generate inhalable aerosol based on heating the aerosol-generating article; and/or
wherein the at least one threshold value is less than about 95 mAh, preferably between about 60 mAh and 85 mAh.

Example 39: The aerosol-generating device according to any one of the preceding examples, wherein the control circuitry is configured to determine, based on historic data indicative of an operation of the aerosol-generating device to generate aerosol, a maximum number of usage sessions the aerosol-generating device is usable with a fully charged energy storage, and to adjust the at least one threshold value based on the determined maximum number of usage sessions.

Example 40: The aerosol-generating device according to any one of the preceding examples, wherein the control circuitry is configured to indicate the determined maximum number of usage sessions to the user based on controlling a user interface of the aerosol-generating device.

Example 41: The aerosol-generating device according to any one of the preceding examples, wherein the control circuitry is configured to enable or allow use of the aerosol-generating device for a subsequent usage session independent of a number of previous usage sessions, the aerosol-generating device has been used since a previous charging process.

Example 42: The aerosol-generating device according to any one of the preceding examples, wherein the at least one threshold value is adjustable based on a user input; and/or wherein the control circuitry is configured to alter or modify the at least one threshold value based on a user input.

Example 43: The aerosol-generating device according to any one of the preceding examples, wherein the at least one threshold value is adjustable based on a user input indicative of a number of usage sessions per charging cycle of the energy storage and/or indicative of a duration of one or more usage sessions, for example a single usage session.

Example 44: The aerosol-generating device according to any one of the preceding examples, wherein the control circuitry is configured to compute the at least one threshold value based on historic data collected in one or more past usage sessions.

Example 45: The aerosol-generating device according to any one of the preceding examples, wherein the control circuitry is configured to compute the at least one threshold value based on or based on computing a current value of a running average of the energy consumption per usage session and/or duration of a usage session, for example based on processing the corresponding data elements of the historic data

Example 46: The aerosol-generating device according to any one of the preceding examples, wherein the control circuitry is configured to calculate the current value of the running average of the energy consumption per usage session and/or duration of a usage session based on a preceding or previous value of the running average of the energy consumption per usage session and/or duration of a usage session, as for example stored in a data storage or RAM of the aerosol-generating device.

Example 47: The aerosol-generating device according to any one of the preceding examples, wherein the control circuitry is configured to compute the at least one threshold value based on computing a plurality of values of running averages of the amount of energy consumed during a usage session, energy consumption per usage session and/or of the duration of a usage session, wherein different running averages differ in terms of the number of usage sessions considered for the particular running average.

Example 48: The aerosol-generating device according to any one of the preceding examples, wherein the control circuitry is configured to compute the at least one threshold value based on computing a first value of a first running average for the energy consumed per usage session and/or duration of a usage session based on a first number of previous usage sessions, and a second value of a second running average for the energy consumed per usage session and/or duration of a usage session based on a second number of previous usage sessions, wherein the first number of previous usage sessions is different than the second number of previous usage sessions. In particular, the first number of previous usage sessions may be smaller than the second number of previous usage sessions.

Example 49: The aerosol-generating device according to example 48, wherein the first number of previous usage sessions is smaller than the second number of previous usage sessions.

Example 50: The aerosol-generating device according to any one of the preceding examples, wherein the control circuitry is configured to determine, based on historic data indicative of an operation of the aerosol-generating device in a pause mode, an amount of energy used during a single usage pause, and to adjust the at least one threshold value based on the determined amount of energy.

Example 51: The aerosol-generating device according to any one of the preceding examples, wherein the at least one threshold value is indicative of a threshold energy required for operating the aerosol-generating device during at least one usage pause interrupting a usage session; and/or wherein the at least one threshold value ranges from about 2 mAh to about 65 mAh.

Example 52: The aerosol-generating device according to any one of the preceding examples, wherein the at least one threshold value is received from a computing device or receiving device communicatively couplable to the aerosol-generating device.

Example 53: The aerosol-generating device according to any one of the preceding examples, wherein the at least one threshold value is indicative of a time period or duration of a single usage session.

Example 54: The aerosol-generating device according to any one of the preceding examples, wherein the at least one threshold value is indicative of a time period or duration of a single usage pause.

Example 55: The aerosol-generating device according to any one of the preceding examples, wherein the at least one threshold value is indicative of an average energy consumption per usage session.

Example 56: The aerosol-generating device according to any one of the preceding examples, wherein the at least one threshold value is indicative of an average energy consumption per usage pause interrupting a usage session.

Example 57: The aerosol-generating device according to any one of the preceding examples, wherein the control circuitry is configured to compute the at least one threshold value based on historic data and to store the computed at least one threshold value in a data storage of the aerosol-generating device.

Example 58: The aerosol-generating device according to any one of the preceding examples, wherein the control circuitry is configured to retrieve historic data from a data storage of the aerosol-generating device and/or from an external data source, the historic data being indicative of one or more of a number of usage sessions the aerosol-generating device has been operated to generate aerosol, a number of usage pauses the aerosol-generating device has been operated in a pause mode interrupting one or more usage sessions, a duration of one or more usage sessions, a duration of one or more usage pauses interrupting one or more usage sessions, energy consumption during one or more usage sessions, energy consumption during one or more usage pauses, total energy consumption, charging data related to charging of the energy storage in one or more charging cycles, remaining energy in the energy storage before re-charging in one or more charging cycles, and energy consumption per usage session.

Example 59: The aerosol-generating device according to any one of the preceding examples, wherein the control circuitry is configured to determine a health status of the energy storage based on historic data indicative of an operation of the aerosol-generating device to generate aerosol.

Example 60: The aerosol-generating device according to any one of the preceding examples, wherein the control circuitry is configured to compute the at least one threshold value based on data received from a computing device or a receiving device communicatively couplable to the aerosol-generating device.

Example 61: The aerosol-generating device according to any one of the preceding examples, wherein the control circuitry is configured to compute the at least one threshold value based on historic data related to one or more previous usage sessions.

Example 62: The aerosol-generating device according to any one of the preceding examples, wherein the control circuitry is configured to compute an average energy consumption of energy consumed during a plurality of usage sessions based on historic data indicative of an operation of the aerosol-generating device to generate aerosol.

Example 63: The aerosol-generating device according to any one of the preceding examples, further comprising a heating element coupled to the energy storage and configured to heat the aerosol-generating article.

Example 64: The aerosol-generating device according to any one of the preceding examples, wherein the aerosol-generating device is couplable to an aerosol-generating article including at least one heating element; and wherein the energy storage is configured to supply electrical energy to the at least one heating element of the aerosol-generating article to generate aerosol.

Example 64a: The aerosol-generating device according to any one of the preceding examples, wherein the aerosol comprises nicotine.

Example 65: An aerosol-generating system, comprising:
an aerosol-generating device according to any one of the preceding examples, and
an aerosol-generating article couplable or coupled to the aerosol-generating device to generate aerosol from at least a portion of the aerosol-generating article, optionally based on heating at least a portion of the aerosol-generating article.

Example 65a: The aerosol-generating system according to example 65 wherein the aerosol-generating article comprises an aerosol-generating substate comprising nicotine.

Example 66: Use of an aerosol-generating device according to any one of examples 1a to 64a or an aerosol-generating system according to any one of examples 65 and 65a to generate aerosol.

Example 67a: A method of operating an aerosol-generating device including an energy storage configured to supply electrical energy to control circuitry operatively for generating aerosol from an aerosol-generating article, the method comprising:
determining, with the control circuitry, a storage status of the energy storage indicative of an amount of electrical energy currently stored and/or storable in the energy storage;
   evaluating the determined storage status with respect to at least one threshold value associated with at least one device function of the aerosol-generating device, in particular associated with heating of the aerosol-generating article; and
enabling or disabling, based on the evaluation, at least one device function, for example a main heating function and/or at least one auxiliary device function.

Example 67b: A method of operating an aerosol-generating device including an energy storage configured to supply electrical energy to control circuitry for generating aerosol from an aerosol-generating article, the method comprising:
determining, with the control circuitry, a storage status of the energy storage indicative of at least one of an amount of electrical energy currently stored and an amount of electrical energy currently storable in the energy storage;
   evaluating the determined storage status with respect to at least one threshold value, wherein the at least one threshold value correlates with a threshold energy required for performing a main heating function of the aerosol-generating device for heating the aerosol-generating article at or above a predetermined heating temperature to generate aerosol in at least one usage session, and for performing at least one auxiliary device function of the aerosol-generating device different than the main heating function; and
enabling or disabling, based on the evaluation, at least one of the main heating function and the at least one auxiliary device function

Example 67c: A method of operating an aerosol-generating device including an energy storage configured to supply electrical energy to control circuitry for generating aerosol from an aerosol-generating article, the method comprising:
determining, with the control circuitry, a storage status including a health status of the energy storage indicative of an amount of electrical energy currently storable in the energy storage;
   evaluating the determined storage status with respect to at least one threshold value associated with at least one device function of the aerosol-generating device; and
enabling or disabling, based on the evaluation, at least one device function.

Example 67d: A method of operating an aerosol-generating device including an energy storage configured to supply electrical energy to control circuitry for generating aerosol from an aerosol-generating article, the method comprising:
determining, with the control circuitry, a storage status of the energy storage indicative of at least one of an amount of electrical energy currently stored and an amount of electrical energy currently storable in the energy storage;
   evaluating the determined storage status with respect to at least one threshold value associated with at least one device function of the aerosol-generating device, wherein the at least threshold value is adjustable; and
enabling or disabling, based on the evaluation, at least one device function.

Example 67e: The method according to any one examples 67a to 67d wherein the energy storage is configured to supply electrical energy to a heating element to heat an aerosol-generating article couplable to the aerosol-generating device.

Example 67f: The method according to any one of examples 67a to 67e wherein the control circuitry is operatively coupled to the energy storage.

Example 67e: The method according to any one of examples 67a to 67d, wherein the energy storage is configured to supply electrical energy to one or more aerosol generators for generating aerosol from at least a portion of an aerosol-generating article couplable to the aerosol-generating device.

Example 67f: The method according to any one of examples 67a to 67e, wherein the energy storage is configured to supply electrical energy to at least one heating element to heat at least a portion of the aerosol-generating article couplable to the aerosol-generating device.

Example 67g: The method according to any one of examples 67a to 67f, wherein the control circuitry is operatively coupled to the energy storage.

Example 68: The method according to any one of examples 67a to 67g, wherein determining the storage status includes determining at least one of a current energy level of electrical energy stored in the energy storage, and a health status of the energy storage indicative of the amount of electrical energy currently storable in the energy storage.

Example 69: The method according to any one of examples 67a to 68, wherein evaluating the determined storage status includes comparing the determined storage status to the at least one threshold value.

Example 70: The method according to any one of examples 67a to 69, wherein the at least one device function is enabled by the control circuitry upon determining, based on the evaluation, that a current energy level of electrical energy stored and/or storable in the energy storage is sufficient for operating the aerosol-generating device during at least one usage session to generate inhalable aerosol based on heating the aerosol-generating article to a temperature at or above a predetermined heating temperature.

Example 71: The method according to any one of examples 67a to 70, further comprising enabling a main heating function upon determining, based on the evaluation, that a current energy level of electrical energy stored and/or storable in the energy storage is sufficient for performing the main heating function during at least one usage session to generate inhalable aerosol based on heating the aerosol-generating article to a temperature at or above a predetermined heating temperature.

Example 72: The method according to any one of examples 67a to 71, further comprising enabling the at least one device function upon determining, based on the evaluation, that a current energy level of electrical energy stored and/or storable in the energy storage is sufficient for completing a current usage session and/or for completing a predefined number of usage sessions to generate inhalable aerosol based on heating the aerosol-generating article to a temperature at or above a predetermined heating temperature.

Example 73: The method according to any one of examples 67a to 72, further comprising enabling the at least one device function upon determining, based on the evaluation, that a current energy level of electrical energy stored and/or storable in the energy storage is sufficient for operating the aerosol-generating device during at least one usage pause in a pause mode interrupting a usage session.

Example 74: The method according to any one of examples 67a to 73, further comprising enabling at least one auxiliary device function upon determining, based on the evaluation, that a current energy level of electrical energy stored and/or storable in the energy storage is sufficient for performing the at least one auxiliary device function of the aerosol-generating device.

Example 75: The method according to any one of examples 67a to 74, further comprising enabling at least one of a main heating function of the aerosol-generating device to generate aerosol in at least one usage session and at least one auxiliary device function of the aerosol-generating device different than the main heating function and/or different than heating the aerosol-generating device during a usage session to generate aerosol based on heating the aerosol-generating article to a temperature at or above a predetermined heating temperature.

Example 76: The method according to any one of examples 67a to 75, further comprising enabling one or more auxiliary device functions including one or more of:
operating the aerosol-generating device at reduced energy consumption with respect to operation during a usage session to generate aerosol;
   heating the at least one heating element and/or the aerosol-generating article to a temperature below a heating temperature sufficient to generate aerosol;
   heating the at least one heating element and/or the aerosol-generating article to a temperature above room temperature and below a heating temperature sufficient to generate aerosol;
   heating the at least one heating element and/or the aerosol-generating article to a temperature above room temperature and below a heating temperature sufficient to generate aerosol for a predefined period of time;
   operating a user interface of the aerosol-generating device;
operating a communication circuitry of the aerosol-generating device for communicatively coupling the aerosol-generating device to a computing device or receiving device;
operating a sensor of the aerosol-generating device; and
operating a biometric sensor of the aerosol-generating device for user authentication.

Example 77: The method according to any one of examples 67a to 76, further comprising enabling a) a main heating function of the aerosol-generating device in at least one usage session to generate aerosol, and b) at least one auxiliary device function of the aerosol-generating device different than the main heating function of the aerosol-generating device, upon determining, based on the evaluation, that a current energy level of electrical energy stored and/or storable in the energy storage is sufficient for performing the main heating function and the at least one auxiliary device function of the aerosol-generating device.

Example 78: The method according to any one of examples 67a to 77, further comprising triggering re-charging of the energy storage based on the evaluation of the determined storage status with respect to the at least one threshold value.

Example 79: The method according to any one of examples 67a to 78, wherein determining the storage status includes determining a current energy level of electrical energy stored and/or storable in the energy storage relative to a current maximum capacity of the energy.

Example 80: The method according to any one of examples 67a to 79, further comprising determining an initial maximum capacity of the energy storage.

Example 81: The method according to any one of examples 67a to 80, further comprising determining whether the energy storage has been charged to a predefined charging stage in a previous charging event.

Example 82: The method according to any one of examples 67a to 81, wherein determining the storage status includes determining an age of the energy storage.

Example 83: The method according to any one of examples 67a to 82, further comprising determining one or more of a current maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated to generate aerosol, charging data related to charging of the energy storage in one or more charging cycles, and a total energy consumption of the aerosol-generating device.

Example 84: The method according to any one of examples 67a to 83, wherein determining the storage status includes determining a current maximum capacity of the energy storage indicative of a maximum amount of energy storable in the energy storage and determining a current energy level or amount of electrical energy currently stored in the energy storage.

Example 85: The method according to any one of examples 67a to 84, wherein the at least one threshold value includes a first threshold value for a current energy level or amount of energy available in the energy storage, and a second threshold value for a current maximum capacity of the energy storage indicative of a maximum amount of energy currently storable in the energy storage; and wherein the method further comprises:
comparing a determined current energy level to the first threshold value, and
comparing a determined current maximum capacity of the energy storage to the second threshold value.

Example 86: The method according to any one of examples 67a to 85, further comprising adjusting the at least one threshold value.

Example 87: The method according to any one of examples 67a to 86, further comprising adjusting the at least one threshold value based on historic data indicative of a past usage of the aerosol-generating device and/or indicative of a consumption behaviour of a user.

Example 88: The method according to any one of examples 67a to 87, further comprising computing the at least one threshold value, based on historic data indicative of an operation of the aerosol-generating device to generate aerosol, an amount of energy used in a one or more usage sessions, preferably a single usage session, and to adjust the at least one threshold value based on the determined amount of energy used in the one or more usage sessions, preferably the single usage session.

Example 89: The method according to any one of examples 67a to 88, further comprising determining, based on historic data indicative of an operation of the aerosol-generating device to generate aerosol, a maximum number of usage sessions the aerosol-generating device is usable with a fully charged energy storage, and to adjust the at least one threshold value based on the determined maximum number of usage sessions.

Example 90: The method according to any one of examples 67a to 89, further comprising indicating the determined maximum number of usage sessions to the user based on controlling a user interface of the aerosol-generating device.

Example 91: The method according to any one of examples 67a to 90, further comprising enabling or allowing use of the aerosol-generating device for a subsequent usage session independent of a number of previous usage sessions, the aerosol-generating device has been used since a previous charging process.

Example 92: The method according to any one of examples 67a to 91, wherein the at least one threshold value is adjusted based on a user input.

Example 93: The method according to any one of examples 67a to 92, further comprising computing the at least one threshold value based on historic data collected in one or more past usage sessions.

Example 94: The method according to any one of examples 67a to 93, further comprising determining, based on historic data indicative of an operation of the aerosol-generating device in a pause mode, an amount of energy used during one or more usage pauses, preferably during a single usage pause, and adjusting the at least one threshold value based on the determined amount of energy.

Example 95: The method according to any one of examples 67a to 94, further comprising receiving the at least one threshold value from a computing device or receiving device communicatively couplable to the aerosol-generating device.

Example 96: The method according to any one of examples 67a to 95, further comprising computing the at least one threshold value based on historic data and storing the computed at least one threshold value in a data storage of the aerosol-generating device.

Example 97: The method according to any one of examples 67a to 96, further comprising processing historic data indicative of one or more of a number of usage sessions the aerosol-generating device has been operated to generate aerosol, a number of usage pauses the aerosol-generating device has been operated in a pause mode interrupting one or more usage sessions, a duration of one or more usage sessions, a duration of one or more usage pauses interrupting one or more usage sessions, energy consumption during one or more usage sessions, energy consumption during one or more usage pauses, total energy consumption, charging data related to charging of the energy storage in one or more charging cycles, remaining energy in the energy storage before re-charging in one or more charging cycles, and energy consumption per usage session.

Example 98: The method according to any one of examples 67a to 97, further comprising determining a health status of the energy storage based on historic data indicative of an operation of the aerosol-generating device to generate aerosol.

Example 99: The method according to any one of examples 67a to 98, further comprising computing the at least one threshold value based on data received from a computing device or a receiving device communicatively couplable to the aerosol-generating device.

Example 100: The method according to any one of examples 67a to 99, further comprising computing the at least one threshold value based on historic data related to one or more previous usage sessions.

Example 101: The method according to any one of examples 67a to 100, further comprising computing an average energy consumption of energy consumed during a plurality of usage sessions based on historic data indicative of an operation of the aerosol-generating device to generate aerosol.

Example 101a: The method according to any one of examples 67a to 101, wherein the aerosol comprises nicotine.

Example 101b: The method according to any one of examples 67a to 110a, wherein the aerosol-generating article comprises an aerosol-generating substrate comprising nicotine.

Example 102: A computer program, which when executed by an aerosol-generating device or an aerosol-generating system, instructs the aerosol-generating device or system to perform steps of the method according to any one of examples 67a to 101b.

Example 103: A non-transitory computer-readable medium storing a computer program according to example 102.

Examples will now be further described with reference to the Figures in which:

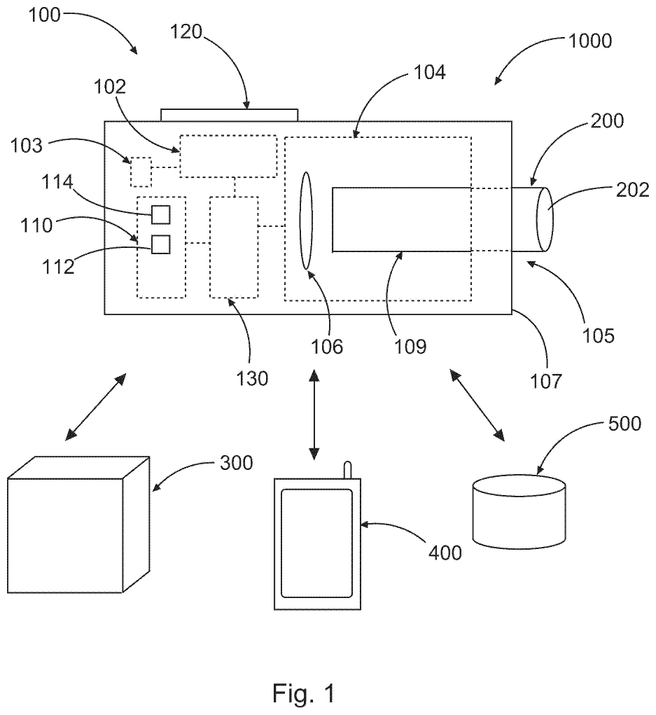

FIG. 1 shows an aerosol-generating device and aerosol-generating system;

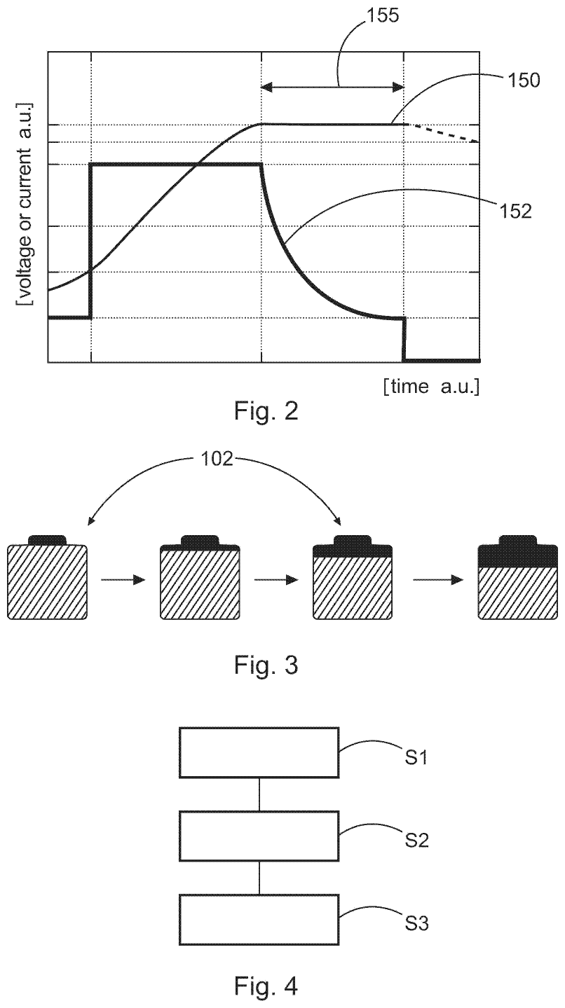

FIG. 2 shows an exemplary charging profile of an energy storage of an aerosol-generating device;

FIG. 3 illustrates aging of an energy storage of an aerosol-generating device; and

FIG. 4 illustrates one or more methods of operating one or more aerosol-generating devices.

The Figures are schematic only and not true to scale. In principle, identical or like parts, elements and/or steps are provided with identical or like reference numerals in the figures.

FIG. 1 shows an exemplary aerosol-generating device 100. The aerosol-generating device 100 of FIG. 1 is exemplary shown as part of an aerosol-generating system 1000, which includes optional components, such as an aerosol-generating article 200, a companion device 300, a mobile device 400, and a computing device 500. It is noted that the aerosol-generating device 100 may be operated as standalone device 100 without any of the optional components 200, 300, 400, 500 of the system 1000.

The aerosol-generating device 100 includes one or more energy storages 102 for storing electrical energy and/or for providing electrical energy to generate aerosol. The one or more energy storages may be one or more batteries (e.g. a lithium-ion battery). When the energy storage(s) 102 is/are a battery/batteries the cathode material may comprise lithium-cobalt-oxide (LCO), lithium-manganese-oxide (LMO), lithium-nickel-manganese-cobalt-oxide (NMC), lithium-iron-phosphate (LFP), and/or lithium-nickel-cobalt-aluminium-oxide (NCA). The anode material may comprise carbon (e.g. graphite), silicon and/or lithium-titanate-oxide (LTO).

The exemplary aerosol-generating device 100 shown in FIG. 1 includes at least a part of a heating circuit 104 with at least one heating element 106 for heating at least a part of an aerosol-generating article 200 couplable to the aerosol-generating device 100. It should be noted that the heating circuit 104 and heating element 106 are optional only. Alternatively or additionally, at least a part of or the entire heating circuit 104, heating arrangement 104 and/or heating element 106 may be integrated or arranged in the aerosol-generating article 200.

Also, it should be noted that the heating element 106 is merely for illustrative purposes shown in FIG. 1 as an inductive coil configured to inductively heat at least a part of the aerosol-generating article 200, for example a susceptor material arranged in an aerosol-generating substrate 202 of the aerosol-generating article 200. Alternatively or additionally, the at least one heating element 106 may be configured for one or more of resistive heating and microwave heating.

Further, it should be noted that the aerosol-generating article 200 is only exemplary shown in FIG. 1 as having a stick-like or tubular shape and as being at least partially insertable through an opening 105 of a housing 107 of the aerosol-generating device 100, for example into a heating chamber 109 of the aerosol-generating device 100. In other exemplary designs, the aerosol-generating article 200 may be shaped as container or cartridge that may be fixedly integrated in the aerosol-generating device 100 or that may be couplable to the device 100.

The aerosol-generating device 100 further comprises control circuitry 110 or device control circuitry 110 operatively coupled to the energy storage 102. The control circuitry 110 may optionally include one or more processors 112 for data processing. The control circuitry 110 may comprise a microcontroller comprising a processor, memory and input/output means.

Further optionally, the aerosol-generating device 100 and/or the control circuitry 110 includes a data storage 114 for storing data, such as for example a storage status of the energy storage, one or more threshold values associated with at least one device function and/or historic data indicative of one or more past usage sessions and/or use pauses, as described in more detail hereinabove and hereinbelow.

Alternatively or additionally, software instructions may be stored in the data storage 114, which when executed by the control circuitry 112 instruct the aerosol-generating device 100 to perform one or more functions of the device 100, as described hereinabove and hereinbelow. The aerosol-generating device 100 optionally includes a user interface 120 for receiving one or more user inputs from a user, for example to operate the aerosol-generating device 100 to generate aerosol. The user interface 120 is exemplary shown as button in FIG. 1. Any other type of user interface 120, such as an acoustic interface, a haptic interface, a touch interface, a display, an arrangement of one or more LEDs or the like can be optionally included in the aerosol-generating device 100 in the alternative or in addition.

Further optionally, the aerosol-generating device 100 includes a communication interface or circuitry 130 for communicatively coupling the aerosol-generating device 100 to one or more optional components of the aerosol-generating system 1000, in particular to one or more of the companion device 300, the mobile device 400, and the computing device 500.

One or more communication interface types or communication protocols may be implemented in the aerosol-generating device 100 and its communication interface or circuitry 130. In particular, the communication interface or circuitry 130 may be configured for one or both wired and wireless communication with one or more of the computing device 500, the mobile device 400 and the companion device 300. For example, the communication interface 130 may be based on one or more of a BUS communication, a cable communication, a Bluetooth communication, a Wireless Local Area Network communication, an infrared communication, a nearfield communication, an internet communication or any other suitable type of communication or communication protocol.

The aerosol-generating device 100 may optionally be coupled to, for example at least partly inserted into, the companion device 300 for charging the energy storage 102 and/or for storing the aerosol-generating device 100. Charging may, for example, be based on inductive charging or via electrical connections.

The aerosol-generating device 100 and/or the control circuitry 110 is configured to supply electrical energy to the at least one heating element 106 to heat at least a portion of the aerosol-generating article 200.

Further, the device control circuitry 110 may be configured to determine a storage status of the energy storage indicative of at least one of an amount of electrical energy currently stored and an amount of electrical energy currently storable in the energy storage 102. Further, the control circuitry 110 may be configured to evaluate the determined storage status with respect to at least one threshold value associated with at least one device function of the aerosol-generating device 110, for example associated with heating of the aerosol-generating article. Further, the control circuitry 110 may be configured to enable or disable, based on the evaluation, at least one device function of the aerosol-generating device 100.

Alternatively or additionally, the control circuitry 110 may be configured to determine a storage status of the energy storage 102 indicative of at least one of an amount of electrical energy currently stored and an amount of electrical energy currently storable in the energy storage, to evaluate the determined storage status with respect to at least one threshold value, wherein the at least one threshold value correlates with a threshold energy required for performing a main heating function of the aerosol-generating device 100 for heating the aerosol-generating article 200 at or above a predetermined heating temperature to generate aerosol in at least one usage session, and for performing at least one auxiliary device function of the aerosol-generating device 100 different than the main heating function. The control circuitry 11 may further be configured to enable or disable, based on the evaluation, at least one of the main heating function and the at least one auxiliary device function of the aerosol-generating device 100.

Alternatively or additionally, the control circuitry 110 may be configured to determine a storage status including a health status of the energy storage 102 indicative of an amount of electrical energy currently storable in the energy storage, to evaluate the determined storage status with respect to at least one threshold value associated with at least one device function of the aerosol-generating device 100, and to enable or disable, based on the evaluation, at least one device function of the aerosol-generating device 100.

Alternatively or additionally, the control circuitry 110 may be configured to determine a storage status of the energy storage indicative of at least one of an amount of electrical energy currently stored and an amount of electrical energy currently storable in the energy storage 102, evaluate the determined storage status with respect to at least one threshold value associated with at least one device function of the aerosol-generating device 100, wherein the at least threshold value is adjustable. Further, the control circuitry 100 may be configured to enable or disable, based on the evaluation, at least one device function of the aerosol-generating device 100.

In the following, various exemplary designs and configurations of the aerosol-generating device 100 are described and summarized. In an example, the the determined storage status includes at least one of a current energy level of electrical energy stored in the energy storage, and a health status of the energy storage indicative of an amount of electrical energy currently storable in the energy storage. For instance, the control circuitry 110 may compare the determined storage status to the threshold value.

For instance, the at least one threshold value may be indicative of a threshold energy required for operating the aerosol-generating device 100 during at least one usage session to generate inhalable aerosol based on heating the aerosol-generating article 100 to a temperature at or above a predetermined heating temperature to generate aerosol. Alternatively or additionally, the at least one threshold value may be indicative of a threshold energy required for performing a main heating function of the aerosol-generating device 100 during at least one usage session, the main heating function involving supplying electrical energy to the at least one heating element 106 to heat at least a part of the aerosol-generating article 200 to a temperature at or above a predetermined heating temperature to generate aerosol.

For example, the at least one threshold value may be indicative of a threshold energy required for completing a current usage session to generate inhalable aerosol based on heating the aerosol-generating article 200 to a temperature at or above a predetermined heating temperature to generate aerosol.

Alternatively or additionally, the at least one threshold value may be indicative of a threshold energy required for operating the aerosol-generating device 100 during at least one usage pause in a pause mode interrupting a usage session.

Alternatively or additionally, the at least one threshold value may be indicative of a threshold energy required for performing at least one auxiliary device function of the aerosol-generating device. The auxiliary device function may be non-related to or differ from heating the aerosol-generating device during a usage session to generate aerosol based on heating the aerosol-generating article to a temperature at or above a predetermined heating temperature to generate aerosol. Alternatively or additionally, the auxiliary device function may be associated with a pause mode interrupting a usage session.

Optionally, the auxiliary device function may include one or more of operating the aerosol-generating device at reduced energy consumption with respect to operation during a usage session to generate aerosol; heating the at least one heating element and/or the aerosol-generating article to a temperature below a heating temperature sufficient to generate aerosol; heating the at least one heating element and/or the aerosol-generating article to a temperature above room temperature and below a heating temperature sufficient to generate aerosol; heating the at least one heating element and/or the aerosol-generating article to a temperature above room temperature and below a heating temperature sufficient to generate aerosol for a predefined period of time; activating or deactivating haptic control of the aerosol-generating device; operating a user interface of the aerosol-generating device; operating a communication circuitry of the aerosol-generating device for communicatively coupling the aerosol-generating device to a computing device or receiving device; operating a sensor of the aerosol-generating device; and operating a biometric sensor of the aerosol-generating device for user authentication.

In an example, the at least one threshold value correlates with a threshold energy required for a) performing a main heating function of the aerosol-generating device in at least one usage session to generate aerosol, and b) for performing an auxiliary device function of the aerosol-generating device different than the main heating function of the aerosol-generating device. The control circuitry 110 may be configured to enable a) a main heating function of the aerosol-generating device in at least one usage session to generate aerosol, and b) at least one auxiliary device function of the aerosol-generating device different than the main heating function of the aerosol-generating device, upon determining, based on the evaluation, that a current energy level of electrical energy stored and/or storable in the energy storage is sufficient for performing the main heating function and the at least one auxiliary device function of the aerosol-generating device.

Optionally, the control circuitry 110 may be configured to trigger or request re-charging of the energy storage 102 based on the evaluation of the determined storage status with respect to the at least one threshold value.

Further, the control circuitry 110 may be configured to determine a health status of the energy storage 102. Optionally, the at least one threshold value may be indicative of a threshold health status and/or threshold age of the energy storage 102.

Further, the control circuitry 110 may be configured to determine a health status of the energy storage 102 based on determining one or more of a current maximum capacity of the energy storage 102, a current maximum capacity of the energy storage 102 with respect to an initial maximum capacity of the energy storage, a number of usage sessions the aerosol-generating device 100 has been used to generate aerosol, an operation time the aerosol-generating device 100 has been operated to generate aerosol, charging data related to charging of the energy storage 102 in one or more charging cycles, and a total energy consumption of the aerosol-generating device 100.

In yet another example, the control circuitry 110 may be configured to determine a current maximum capacity of the energy storage 102 indicative of a maximum amount of energy storable in the energy storage 102 and a current energy level or amount of electrical energy currently stored in the energy storage 102.

Optionally, the control circuitry 110 may be configured to adjust the at least one threshold value, for example based on one or more user inputs and/or based on historic data indicative of a past usage of the aerosol-generating device and/or indicative of a consumption behavior of a user.

The control circuitry 110 may be configured to compute, based on historic data indicative of an operation of the aerosol-generating device to generate aerosol, an amount of energy used in a single usage session, and to adjust the at least one threshold value based on the determined amount of energy used in the single usage session. For instance, the at least one threshold value may be indicative of a threshold energy required for operating the aerosol-generating device 100 during at least one usage session to generate inhalable aerosol based on heating the aerosol-generating article, and wherein the at least one threshold value may be less than about 95 mAh, preferably between about 60 mAh and 85 mAh.

The control circuitry 110 may further be configured to determine, based on historic data indicative of an operation of the aerosol-generating device 100 to generate aerosol, a maximum number of usage sessions the aerosol-generating device is usable with a fully charged energy storage 102, and to adjust the at least one threshold value based on the determined maximum number of usage sessions.

Optionally, the control circuitry 110 may be configured to actuate the user interface 120 to indicate the determined maximum number of usage sessions to the user.

Alternatively or additionally, the control circuitry 110 may be configured to determine, based on historic data indicative of an operation of the aerosol-generating device 100 in a pause mode, an amount of energy used during a single usage pause, and to adjust the at least one threshold value based on the determined amount of energy. For instance, the at least one threshold value may be indicative of a threshold energy required for operating the aerosol-generating device 100 during at least one usage pause interrupting a usage session, and wherein the at least one threshold value may range from about 2 mAh to about 65 mAh.

With continued reference to FIG. 1 and subsequent FIGS. 2 and 3, illustrative exemplary features of exemplary aerosol-generating devices 100 according to the disclosure are summarized, which can be implemented alone or in combination with any of the functions of the aerosol-generating device 100 described hereinabove. FIG. 2 shows an exemplary charging profile of an energy storage 102 of an aerosol-generating device 100, and FIG. 3 illustrates aging of an energy storage 102 of an aerosol-generating device 100.

The aerosol-generating device 100 includes an energy storage 102 and a control circuitry 110, as described with reference to FIG. 1. One of the aerosol-generating device's 100 device function or auxiliary device function may be the pause mode, which may allow a user to interrupt an ongoing usage session and resume it without substantial degradation of the aerosol-generating article 200, for example due to condensation of vapor in the device 100 or article 200. As described in detail above, the device 100 may be switched by the user, for example based on actuating the user interface 120, from the aerosol-releasing mode, in which the main heating function of the device 100 may be active and the heating element 106 may be heated to the predetermined heating temperature, into the pause mode. During the usage pause or in the pause mode, the temperature of the heating element 106 may be kept at a temperature above room temperature and below the predetermined heating temperature.

The control circuitry 110 of the device 100 may be configured to determine, based on evaluating a determined storage status of the energy storage 102, with at least one threshold value associated with the pause mode or a device function, whether or not the pause mode should be allowed or enabled at the device 100.

Generally, pause mode may allow users to pause in between puffs during a usage session. The pause can, for example range between a few seconds up to a couple of minutes, for example eight or ten minutes. During usage pause, the temperature of the substrate 202, article 200, and/or heating element 106 may be kept at certain level, for example the second temperature level, as described hereinabove. When the pause mode is terminated by the user or otherwise ends, the temperature would raise to the predetermined heating temperature or first temperature level allowing the user to continue the experience or usage session. As energy will be consumed during the pause mode, to ensure the energy storage 102 would have sufficient energy to complete the experience or usage session, one or more requirements, criteria, or preconditions may be considered and should preferably be fulfilled for activation of the pause mode. One or more of these requirements can be defined or included in the at least one threshold value that is evaluated with the storage status of the energy storage 102.

For example, at least one threshold value indicative of a current capacity of the energy storage 102 and/or at least one threshold value indicative of a health status of the energy storage 102 may be considered and used by the control circuitry 110 to evaluate the storage status of the energy storage 102.

Particularly, a capacity value of the energy storage 102 or amount of electrical energy currently stored in the energy storage 102 may be, for example, at least 60%, 70%, 75%, 80%, 85%, 90%, 95% and 100% of a current maximum capacity. Preferably, the capacity value or amount of electrical energy currently stored in the energy storage 102 should be at least 90%, 95% and 100% of the current maximum capacity.

Alternatively or additionally, pause mode may be enabled upon determining that the energy storage 102 is fully charged.

In another embodiments, the control circuitry 110 may determine whether a particular state of charge has been reached in a previous charging cycle. FIG. 2 shows an exemplary charging profile of an energy storage 102 of an aerosol-generating device 100, wherein the charging voltage 150 and the charging current 152 profiles are shown in arbitrary units versus charging time in arbitrary units.

In a non-limiting example, the at least one threshold value may be indicative of a predefined charging stage the energy storage has reached in a previous charging event, in particular a constant voltage stage shown by reference numeral 155 in FIG. 2. The constant voltage stage may be at 3.65 V charging voltage. Alternatively or additionally, one or more characteristics related to charging current and charging time may be considered by the control circuitry 110 to evaluate the storage status and/or enable or disable the pause mode.

Alternatively or additionally, the control circuitry 110 may be configured to determine a storage status including a health status or being indicative of an age of the energy storage 102. Generally, an energy storage 102 may over time and the current storage capacity or amount of energy currently storable in the energy storage 102 may decrease over time.

FIG. 3 illustrates aging of an energy storage 102 of an aerosol-generating device 100. The completely filled energy storage 102 to the very left of FIG. 3 may indicate the energy storage's 102 initial capacity or storage capacity. With increasing time, shown by the sequential illustrations from left to right in FIG. 3, the current storage capacity of the energy storage 102 decreases. This may mean that the energy storage 102 loses part of its capability to store energy and/or can store increasingly less energy per fully charged energy storage with increasing time.

The control circuitry 110 may be configured to determine the health status or age of the energy storage 102 based on determining one or more of number of experiences, total energy consumption, time of use and current maximum capacity, a current maximum capacity of the energy storage, a current maximum capacity of the energy storage with respect to an initial maximum capacity of the energy storage, a number of usage sessions the aerosol-generating device has been used to generate aerosol, an operation time the aerosol-generating device has been operated to generate aerosol, charging data related to charging of the energy storage in one or more charging cycles, and a total energy consumption of the aerosol-generating device.

Upon reaching and/or exceeding one or more corresponding threshold values for one or more of the aforementioned quantities, the pause mode and or the. Main heating function may be disabled or prevented.

A current maximum capacity may be larger than at least 40%, 45%, 50%, 55%, 60%, 65% and 70% of the initial capacity of the energy storage 102. If the current maximum capacity falls below for example 50%, 55% and 60% of the initial capacity, then pause mode and/or the main heating function may not be allowed or enabled.

For example, the initial capacity of the energy storage 102 may be about 300 mAh. The current maximum capacity would equal to the initial capacity. After repeating cycling (charging and discharging), the energy storage 102 may age and the maximum capacity may decrease, as shown in FIG. 3. Therein, the capacity value may refer to the capacity available in the energy storage 102.

Determination of availability of the pause mode can be determined or checked, for example, at an end of each charging event or when the user extracts the device 100 from the companion device 300, at the beginning of experience and during the experience or usage session.

Upon determining that the pause mode can be activated or enabled, the control circuitry 110 may actuate the user interface 120, for example indicate or notify the user about the availability of the pause mode. For instance, an LED may indicate availability or unavailability of the pause mode. Alternatively or additionally, availability of the pause mode may be indicated based in changing colour of one or more LEDs or elements of a user interface 120.

Optionally, availability may be shown in response to a user input, for example in response to pressing a button or actuating a user interface 120.

The capacity of the energy storage 102 may be determined or checked at the end of a charging event or when the user extracts the device 100 from the companion device 300.

The control circuitry 110 may optionally simulate a fuel gauge of the aerosol-generating device 100 and/or request a fuel gauge or a charging circuit of the device 100 to check the capacity of the energy storage 102 or energy stored in the energy storage 102, and a current maximum capacity or storage capacity of energy storable in the energy storage 102. The control circuitry 110 may compare the results from the fuel gauge to determine whether the pause mode should be allowed. When the user tries to activate the pause mode (or when the user extracts the device 100 from the companion device 300), the control circuitry 110 may instruct or operate an indictor notifying whether the pause mode is allowed or enabled.

The indicator may, for example, be a visual indicator (e.g., LED light), an audio indicator, a vibration or tactile indicator, a haptic indicator or a combination thereof. Particularly, a pause symbol with two parallel vertical bars, as used in media players, may be shown at the user interface 120.

The health status may be determined or checked at the end of charging or when the user extracts the device 100 from the companion device 300. The control circuitry 110 may request a data storage, storing data or historic data, for example related to the initial capacity of the battery, the number of experiences or usage sessions, a total energy consumption, a time of use or others, as described above. The control circuitry 110 may simulate or request a fuel gauge (of the device 100 for the current maximum capacity of the energy storage 102. The control circuitry 110 may further estimate the health status to determine whether the pause mode should be allowed. When the user tries to activate the pause mode or when the user extracts the device 100 from the companion device 300, the control circuitry 110 may instruct or operate an indictor notifying whether pause mode is allowed or activated. The indicator may, for example, be a visual indicator (e.g., LED light), an audio indicator, a vibration or tactile indicator, a haptic indicator or a combination thereof. Particularly, a pause symbol with two parallel vertical bars, as used in media players, may be shown at the user interface 120.

Alternatively or additionally, an adjustable or variable threshold value may be implemented in the aerosol-generating device 100, as exemplary summarized in the following.

In standard conventional devices a fixed threshold for energy stored in the energy storage 102 or current capacity is considered to determine whether a usage session or experience can be granted. Also, a fixed number of usage sessions, such as two, per fully charged energy storage 102 may be granted only. Hence, a user may be notified by the control circuitry 110 whether there is sufficient capacity based on a standard capacity value. If the remaining capacity in the energy storage is smaller than the standard capacity value, then the device would advise the user to recharge the device. For example, if a remaining capacity is below the standard capacity value, for example 60 mAh, the device needs to be recharged until reaching the standard energy value, for example 95 mAh, to trigger the notification, for example a light, to a user to have additional experience or usage session granted or allowed

However, it was found that different users may have different consumption time for each experience or usage session, and therefore the energy needed per usage session or experience may vary from user to user. Most of the users consume one experience shorter and capacity or energy needed would be less than about 95 mAh, for example about 50-85 mAh. Using an adjustable threshold value for evaluating the storage status, such as the current capacity or energy stored in the energy storage 102, allows to flexibly cope with different needs from different users. Alternatively or additionally, the pause mode can be dynamically enabled or disabled based on an adjustable threshold value.

Rather than based on a standard or fixed capacity value, such as 95 mAh, a threshold capacity value and/or threshold energy value can be used. The threshold capacity value or threshold energy value may be used to determine whether the device will allow the next experience or usage session and/or pause mode. Whether allowing the next experience or usage session may be independent on the number of credits, experiences or usage sessions granted with a fully charged energy storage 102 or since the last charging event.

The aerosol-generating device 100 comprises a user interface 120, for example serving as an indicator, a control circuitry 110 and optionally a fuel gauge connectable to the energy storage 102.

The fuel gauge may be simulated and implemented in the control circuitry 110. The control circuitry 110 may be configured to measure a remaining capacity or energy stored in the energy storage 102. The control circuitry 110 may be configured to compare the remaining capacity with a threshold energy value to check whether the remaining capacity can provide the threshold energy value. The control circuitry 110 may either convert capacity to energy or energy to capacity, to determine whether the remaining capacity is sufficient for the next, for example N+1, experiences or usage sessions; and indicate a result of whether the remaining capacity is sufficient for the next experience using the indicator or user interface 120.

In this way, a warning can be provided to a user of the aerosol generation device 100 when there is only enough battery capacity remaining for the next experience or usage session. This way allow the user to anticipate the need to charge the energy storage 102 before the energy storage 102 runs out, thereby negating the risk of running out of power when using the device 100.

A threshold capacity value or threshold energy value can be obtained in various ways. For instance, (a) fix capacity value 85 mAh; or 0.272 mWh with a nominal voltage of 3.2V may be used, (b) the value may be input by the user via an interactive platform, for example using a computing device 500 or mobile device 400, and/or (c) calculated according to the historic data collected in previous experience(s) and/or usage session(s).

If the remaining capacity or energy is above the threshold value, for example above 85 mAh or above 0.272 mWh, then the control circuitry 110 may allow the next experience or usage session

The interactive platform, for example App, may be provided to the users allowing them to review the usage data or historic data, for example length of each experience or usage session, and/or input the preference duration of experience or usage session, for example seven minutes. Either the interactive platform or the control circuitry 110 of the device 100 may calculate the threshold capacity value or threshold energy value. For the former case, the threshold energy value and/or threshold capacity value may be transmitted to the device 100

Alternatively or additionally, the aerosol generating device 100 may collect historic data, for example, energy consumption in each previous experience or usage session, capacity consumption in each previous experience or usage session, re-charging, time taken to charge, battery level, type of charger, and others, and calculate at least one threshold value, for instance reflecting an adequate amount of energy or capacity needed for one experience or usage session. Therein, a threshold energy value may be computed as average energy consumption and optionally a standard deviation.

Alternatively or additionally, the threshold energy value can be based on the energy consumed in one or more experiences or usage sessions. For example, it can be based on the 1st experience after fully charged the last time. It can also be based on previous two or more experiences or usage sessions. It can also be based on previous sixteen to twenty or even more experiences or usage sessions.

Alternatively or additionally, the aerosol generating device 10 may calculate a threshold capacity value or adequate amount of capacity needed for one experience or usage session. Therein, a threshold capacity value may be computed as average capacity consumption and optionally a standard deviation.

The historic data may be stored in the data storage or memory of the aerosol-generating device 100, for example in the form of records of energy consumed in previous usage sessions and/or capacity consumed in previous usage sessions.

The device 100 may allow the user to pause between puffs in one experience or usage session and switch the device 100 into a pause mode, as described above. During the pause mode, energy or capacity may be consumed because energy or capacity may be used to hold an aerosol-generating substance or article 200 at certain temperature above the room temperature. The energy or capacity for an experience or usage session and the energy or capacity for pausing may be stored separately in the historic data.

For instance, the control circuitry 110 may compute an average energy consumption as the average energy of previous usage sessions, or average energy of the energy for previous usage sessions and for pausing during one or more usage pauses. Alternatively or additionally, the average capacity consumption may be computed as the average capacity of previous usage sessions, or average capacity of the capacity for previous usage sessions and for pausing during one or more usage pauses.

Optionally, the computed threshold values for energy or capacity may be independent to a charging event. Alternatively or additionally, the threshold value for energy or capacity may depend on the charging event. For example, after fully charging the energy storage 102, the user may enjoy the 1st experience and 2nd experience or usage session. The user fully re-charges the battery and enjoys the 3rd experience. Whether the user can enjoy the 4th experience would only depend on the data of the 3rd experience or usage session.

The control circuitry 110 of the device 100 may determine whether a remaining capacity in the energy storage 102 is larger than the threshold energy value. Particularly, the control circuitry 110 may determine whether the next usage session can be granted at an end of a usage session (N). The result may be notified to the user whether the user can have the N+1 experience or usage session granted, for example using the user interface 120.

Where energy is used for comparison, the control circuitry 110 may extract historic data from the memory or data storage of the device 100 and compute a threshold energy value according to the past experiences or usage sessions, as described above. The control circuitry 110 may request or simulate the fuel gauge to determine the remaining capacity or energy stored in the energy storage 102. The control circuitry 110 may optionally convert energy values to capacity values. One or more of current energy or capacity of the energy storage 102 can then be compared to the corresponding one or more threshold values to determine whether the next usage session and/or a pause mode can be allowed. The result can be notified by means of the indicator or user interface 120, for example, visual indicator (e.g., LED light), an audio indicator, vibration or tactile indicator, or a haptic indicator.

Alternatively or additionally, the control circuitry 110 may extract historic data from the memory or data storage and compute a threshold energy value according to the past experiences or usage sessions, for example 16-20 experiences. The control circuitry 110 may simulate or request the fuel gauge to determine the remaining capacity. Optionally the threshold energy value may be converted into a threshold capacity value and compared with the remaining capacity to determine whether the next experience or usage session can be allowed. The result can be notified by means of the indicator or user interface 120, for example, by a visual indicator (e.g., LED light), an audio indicator, vibration or tactile indicator, or a haptic indicator.

Where capacity is used for comparison, the control circuitry 110 may extract historic data from the memory or data storage and compute a threshold capacity value according to the past experiences or usage sessions, for example 16-20 experiences. The control circuitry 110 may simulate or request the fuel gauge to determine the remaining capacity or energy stored. The control circuitry 110 may compare the threshold capacity value and the remaining capacity to determine whether to allow the next experience or usage session. The result can be notified by means of the indicator or user interface 120, for example, by a visual indicator (e.g., LED light), an audio indicator, vibration or tactile indicator, or a haptic indicator.

FIG. 4 illustrates one or more methods of operating one or more aerosol-generating devices, for example an aerosol-generating device 100 described with reference to any one or more of the previous figures.

In step S1, a storage status of the energy storage 102 indicative of an amount of electrical energy currently stored and/or storable in the energy storage 102 is determined with the control circuitry 110.

At step S2, the determined storage status is evaluated with respect to at least one threshold value associated with at least one device function of the aerosol-generating device 100, in particular associated with heating of the aerosol-generating article 200.

Alternatively or additionally, step S2 may comprise evaluating the determined storage status with respect to at least one threshold value, wherein the at least one threshold value correlates with a threshold energy required for performing a main heating function of the aerosol-generating device 100 for heating the aerosol-generating article 200 at or above a predetermined heating temperature to generate aerosol in at least one usage session, and for performing at least one auxiliary device function of the aerosol-generating device 100 different than the main heating function

Alternatively or additionally, step S2 may comprise determining, with the control circuitry 110, a storage status including a health status of the energy storage 102 indicative of an amount of electrical energy currently storable in the energy storage 102.

Alternatively or additionally, step S2 ma comprise evaluating the determined storage status with respect to at least one threshold value associated with at least one device function of the aerosol-generating device 100, wherein the at least threshold value is adjustable.

At step S3, at least one device function, for example a main heating function and/or at least one auxiliary device function, of the aerosol-generating device 100 is enabled or disabled based on the evaluation.

Alternatively or additionally, step S3 may comprise enabling or disabling, based on the evaluation, at least one of the main heating function and the at least one auxiliary device function.

It is noted that any one or more features, functionalities and configurations of the aerosol-generating device 100, as described hereinabove, can be implemented as optional, supplemental or alternative steps in the method of FIG. 4.

For the purpose of the present description and of the appended claims, except where otherwise indicated, all numbers expressing amounts, quantities, percentages, and so forth, are to be understood as being modified in all instances by the term “about” or “substantially”. Also, all ranges include the maximum and minimum points disclosed and include any intermediate ranges therein, which may or may not be specifically enumerated herein. In this context, therefore, a number A is understood as A±20% of A. Within this context, a number A may be considered to include numerical values that are within general standard error for the measurement of the property that the number A modifies. Also, all ranges include the maximum and minimum points disclosed and include any intermediate ranges therein, which may or may not be specifically enumerated herein.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1.-15. (canceled)

16. An aerosol-generating device, comprising:
control circuitry and an energy storage configured to supply electrical energy to the control circuitry for generating aerosol from an aerosol-generating article;
wherein the control circuitry is configured to:
determine a storage status of the energy storage indicative of at least one of an amount of electrical energy currently stored and an amount of electrical energy currently storable in the energy storage,
evaluate the determined storage status with respect to at least one threshold value, wherein the at least one threshold value correlates with a threshold energy required for performing a main heating function of the aerosol-generating device for heating the aerosol-generating article at or above a predetermined heating temperature to generate aerosol in at least one usage session, and for performing at least one auxiliary device function of the aerosol-generating device different than the main heating function, and
enable or disable, based on the evaluation, at least one of the main heating function and the at least one auxiliary device function.

17. The aerosol-generating device according to claim 16, wherein the at least one auxiliary device function is associated with a pause mode interrupting a usage session.

18. The aerosol-generating device according to claim 16, wherein the at least one threshold value is indicative of a threshold energy required for operating the aerosol-generating device during at least one usage pause in a pause mode interrupting a usage session.

19. The aerosol-generating device according to claim 16, wherein the control circuitry is further configured to enable the at least one auxiliary device function upon determining, based on the evaluation, that a current energy level of electrical energy stored and/or storable in the energy storage is sufficient for operating the aerosol-generating device during at least one usage pause in a pause mode interrupting a usage session.

20. The aerosol-generating device according to claim 16, wherein the at least one auxiliary device function includes one or more of:
operating the aerosol-generating device at reduced energy consumption with respect to operation during a usage session to generate aerosol,
heating the at least one heating element and/or the aerosol-generating article to a temperature below a heating temperature sufficient to generate aerosol,
heating the at least one heating element and/or the aerosol-generating article to a temperature above room temperature and below a heating temperature sufficient to generate aerosol,
heating the at least one heating element and/or the aerosol-generating article to a temperature above room temperature and below a heating temperature sufficient to generate aerosol for a predefined period of time,
activating or deactivating haptic control of the aerosol-generating device,
operating a user interface of the aerosol-generating device,
operating a communication circuitry of the aerosol-generating device for communicatively coupling the aerosol-generating device to a computing device or receiving device,
operating a sensor of the aerosol-generating device, and
operating a biometric sensor of the aerosol-generating device for user authentication.

21. The aerosol-generating device according to claim 16, wherein the at least one threshold value correlates with a threshold energy required for operating the aerosol-generating device a) during at least one usage session in an aerosol-releasing mode generating inhalable aerosol based on heating the aerosol-generating article to a temperature at or above the predetermined heating temperature, and b) during a usage pause in a pause mode interrupting the at least one usage session.

22. The aerosol-generating device according to claim 16, wherein the control circuitry is further configured to enable a) the main heating function of the aerosol-generating device in at least one usage session to generate aerosol, and b) the at least one auxiliary device function of the aerosol-generating device different than the main heating function of the aerosol-generating device, upon determining, based on the evaluation, that a current energy level of electrical energy stored and/or storable in the energy storage is sufficient for performing the main heating function and the at least one auxiliary device function of the aerosol-generating device.

23. The aerosol-generating device according to claim 16, wherein the determined storage status includes at least one of a current energy level of electrical energy stored in the energy storage, and a health status of the energy storage indicative of an amount of electrical energy currently storable in the energy storage.

24. The aerosol-generating device according to claim 16, wherein evaluating the determined storage status includes comparing the determined storage status to the at least one threshold value.

25. The aerosol-generating device according to claim 16,
wherein the main heating function involves supplying electrical energy to the at least one heating element to heat at least a part of the aerosol-generating article to a temperature at or above a predetermined heating temperature to generate aerosol, and
wherein the at least one auxiliary device function involves supplying electrical energy to the at least one heating element to heat at least a part of the aerosol-generating article to a temperature below predetermined heating temperature to generate aerosol.

26. The aerosol-generating device according to claim 16, wherein the control circuitry is further configured to determine, based on the evaluation, a number of usage pauses for which the aerosol-generating device is operable.

27. The aerosol-generating device according to claim 26, wherein the control circuitry is further configured to actuate a user interface of the aerosol-generating device to indicate the determined number of usage pauses.

28. The aerosol-generating device according to claim 16, wherein the control circuitry is further configured to trigger or request re-charging of the energy storage based on the evaluation of the determined storage status with respect to the at least one threshold value.

29. The aerosol-generating device according to claim 16, wherein
determining the storage status includes determining a current energy level of electrical energy stored and/or storable in the energy storage relative to a current maximum capacity of the energy.

30. An aerosol-generating system, comprising:
an aerosol-generating device according to claim 16; and
an aerosol-generating article couplable or coupled to the aerosol-generating device to generate aerosol from at least a portion of the aerosol-generating article.
